SEQUENCE LISTING (44 sequences)

SEQ ID NO: 1 - protein, 138 aa, Burkholderia cenocepacia
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 2 - protein, 123 aa, Burkholderia cenocepacia
MYADDFDGEIEIDEVDSLVEFLSRRPAFDANNFVLTFEESGFPQLNIFAKNDIAVVYYMDIGENFVSKGNSASGGTEKFYENKLGGEVDLSKDCVVSKEQMIEAAKQFFATKQRPEQLTWSEL

SEQ ID NO: 3 - protein, 83 aa, Bacillus phage PBS2
TNLSDIIEKETGKQLVIQESILMLPEEVEEVIGNKPESDILVHTAYDESTDENVMLLTSDAPEYKPWALVIQDSNGENKIKML

SEQ ID NO: 4 - protein, 231 aa, Pseudomonas aeruginosa
MTDNDDRIKLEASWKEALREEFDKPYMKQLGEFLRQEKAAGKVIFPPGPLIFNALNTTPLENVKVVIIGQDPYHGPGQAHGLCFSVQPGVPTPPSLQNIYKELNRDLNIPIPNNGYLQRWAEQGVLLLNTSLTVEQAKAGSHANAGWQPFTDRVIEVVNERCERLVFLLWGSHAQSKQKLIDPQRHLILKSAHPSPLSAYRGFLGNGHFSRTNKFLEQNGKTPIDWSLPDL

SEQ ID NO: 5 - protein, 511 aa, Pseudomonas aeruginosa
MRIHVSFIDRVGITQEVLALLGGRNLNLDAVEMVPPNVYIDAPTLSPEVLEELRAALLGIRGVQAMTVVDILPGQRRRLQLDALLAAMADPVLAVDGKGLVLLANPAFSERCGRDPAGERLASLFDDETLEDALVEQGFRLPLREVTFMGQALLLDATPITEGPGEGERHLAGGLLTLYEPNRIGERLAALHHDHAEGFEMLLGDSQPIRTLKTRAQRVAALDAPLLIHGETGTGKELVARGCHALSARHNSPFLALNCAALPENLAESELFGYAPGAFTGAQRGGKPGLLELAHQGTVFLDEIGEMSPYLQAKLLRFLSDGSFRRVGGDREVRVDVRILSATHRNLEKMVAEGSFREDLFYRLNVLSLEVPPLRERGHDILLLARHFMQQACAQIQRPVCRLAPGTYPALLSNRWPGNVRQLQNVIFRAAAICESSLVDIGDLEIAGTAVARQNDGEVGSLEEAVEGFEKALLEKLYVSYPSTRQLAARLQTSHTAIAHRLRKYGIGPRA

SEQ ID NO: 6 - protein, 32 aa, Artificial sequence
  Synthetic
SGGSSGGSSGSETPGTSESATPESSGGSSGGS

SEQ ID NO: 7 - protein, 681 aa, Artificial sequence
  Synthetic
  MISC_FEATURE (1)..(511): Wherein positions 1 to 511 are defined as GcsR domain
  MISC_FEATURE (512)..(543): Wherein positions 512 to 543 are defined as synthetic linker
  MISC_FEATURE (544)..(681): Wherein positions 544 to 681 are defined as DddA domain
MRIHVSFIDRVGITQEVLALLGGRNLNLDAVEMVPPNVYIDAPTLSPEVLEELRAALLGIRGVQAMTVVDILPGQRRRLQLDALLAAMADPVLAVDGKGLVLLANPAFSERCGRDPAGERLASLFDDETLEDALVEQGFRLPLREVTFMGQALLLDATPITEGPGEGERHLAGGLLTLYEPNRIGERLAALHHDHAEGFEMLLGDSQPIRTLKTRAQRVAALDAPLLIHGETGTGKELVARGCHALSARHNSPFLALNCAALPENLAESELFGYAPGAFTGAQRGGKPGLLELAHQGTVFLDEIGEMSPYLQAKLLRFLSDGSFRRVGGDREVRVDVRILSATHRNLEKMVAEGSFREDLFYRLNVLSLEVPPLRERGHDILLLARHFMQQACAQIQRPVCRLAPGTYPALLSNRWPGNVRQLQNVIFRAAAICESSLVDIGDLEIAGTAVARQNDGEVGSLEEAVEGFEKALLEKLYVSYPSTRQLAARLQTSHTAIAHRLRKYGIGPRASGGSSGGSSGSETPGTSESATPESSGGSSGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 8 - DNA, 42 bp, Artificial sequence
  Synthetic
tgaaattcagcaggatcacatatgtacgcagacgatttcgac

SEQ ID NO: 9 - DNA, 44 bp, Artificial sequence
  Synthetic
tcatttcaatatctgtatatctagattacaactcgctccatgtc

SEQ ID NO: 10 - DNA, 49 bp, Artificial sequence
  Synthetic
ggaagcataaatgtaaagcaagcttgcaacctggagaagatggtcgccg

SEQ ID NO: 11 - DNA, 28 bp, Artificial sequence
  Synthetic
tacctccagaggcgcgcggaccgatgcc

SEQ ID NO: 12 - DNA, 28 bp, Artificial sequence
  Synthetic
tccgcgcgcctctggaggtagctccggc

SEQ ID NO: 13 - DNA, 28 bp, Artificial sequence
  Synthetic
gcccgcttcaacaacctcctttcgtggg

SEQ ID NO: 14 - DNA, 28 bp, Artificial sequence
  Synthetic
aggaggttgttgaagcgggctcagccct

SEQ ID NO: 15 - DNA, 46 bp, Artificial sequence
  Synthetic
ttaaggtaccgaattcgagctcgagcaatcccaaggagttcgagcg

SEQ ID NO: 16 - DNA, 45 bp, Artificial sequence
  Synthetic
ggaagcataaatgtaaagcaagcttcggatgtcgtcctgatggac

SEQ ID NO: 17 - DNA, 28 bp, Artificial sequence
  Synthetic
tacctccagagctggcggcatcgaccat

SEQ ID NO: 18 - DNA, 28 bp, Artificial sequence
  Synthetic
tgccgccagctctggaggtagctccggc

SEQ ID NO: 19 - DNA, 28 bp, Artificial sequence
  Synthetic
cgctcatctaacaacctcctttcgtggg

SEQ ID NO: 20 - DNA, 33 bp, Artificial sequence
  Synthetic
aggaggttgttagatgagcgccgttttcgacgc

SEQ ID NO: 21 - DNA, 43 bp, Artificial sequence
  Synthetic
ttaaggtaccgaattcgagctcgagggccgcgtacggttgcgg

SEQ ID NO: 22 - DNA, 43 bp, Artificial sequence
  Synthetic
ggaagcataaatgtaaagcaagctttcgccctgctgctcaacg

SEQ ID NO: 23 - DNA, 29 bp, Artificial sequence
  Synthetic
tacctccagaatcatccgacaggtcgtcg

SEQ ID NO: 24 - DNA, 28 bp, Artificial sequence
  Synthetic
gtcggatgattctggaggtagctccggc

SEQ ID NO: 25 - DNA, 28 bp, Artificial sequence
  Synthetic
cgacctgtcaacaacctcctttcgtggg

SEQ ID NO: 26 - DNA, 35 bp, Artificial sequence
  Synthetic
aggaggttgttgacaggtcgtttcgcaacgctttg

SEQ ID NO: 27 - DNA, 43 bp, Artificial sequence
  Synthetic
ttaaggtaccgaattcgagctcgagcgcgcggagcgaagcagc

SEQ ID NO: 28 - DNA, 57 bp, Artificial sequence
  Synthetic
gataacaatttcagaattcgagctcacgggaggaaagatgacgaatctcagcgacat

SEQ ID NO: 29 - DNA, 51 bp, Artificial sequence
  Synthetic
tcatttcaatatctgtatatctagattagagcatcttgattttgttctcgc

SEQ ID NO: 30 - DNA, 44 bp, Artificial sequence
  Synthetic
gggctagcgaattcgagctcggtaccacgggaggaaagatgtac

SEQ ID NO: 31 - DNA, 44 bp, Artificial sequence
  Synthetic
ctcatccgccaaaacagccaagctttcacaactcgctccatgtc

SEQ ID NO: 32 - DNA, 75 bp, Artificial sequence
  Synthetic
cttctctcatccgccaaaacagccaagctttcatttgtcgtcgtcgtctttgtagtccaactcgctccatgtcag

SEQ ID NO: 33 - DNA, 36 bp, Artificial sequence
  Synthetic
cataaatgtaaagcaagcttggtaccgaggcggact

SEQ ID NO: 34 - DNA, 35 bp, Artificial sequence
  Synthetic
agcccgcttcaggcgcgcgggatgcgcatgcggga

SEQ ID NO: 35 - DNA, 35 bp, Artificial sequence
  Synthetic
caggttcccgcatgcgcatcccgcgcgcctgaagc

SEQ ID NO: 36 - DNA, 39 bp, Artificial sequence
  Synthetic
tcgagctcgagcccggggatccttcgattacccacctgc

SEQ ID NO: 37 - DNA, 38 bp, Artificial sequence
  Synthetic
cataaatgtaaagcaagcttacctgttctaccgcctca

SEQ ID NO: 38 - DNA, 59 bp, Artificial sequence
  Synthetic
cttgccgaggcggttcatttcgatgtcggtgtaagcggccgcggcgcgcggaccgatgc

SEQ ID NO: 39 - DNA, 59 bp, Artificial sequence
  Synthetic
gcggccgcttacaccgacatcgaaatgaaccgcctcggcaagtgaagcgggctcagccc

SEQ ID NO: 40 - DNA, 39 bp, Artificial sequence
  Synthetic
tcgagctcgagcccggggatccgagttcgagcgcttcag

SEQ ID NO: 41 - DNA, 38 bp, Artificial sequence
  Synthetic
cataaatgtaaagcaagcttgaactgaagccggatgtc

SEQ ID NO: 42 - DNA, 59 bp, Artificial sequence
  Synthetic
cttgccgaggcggttcatttcgatgtcggtgtaagcggccgcgctggcggcatcgacca

SEQ ID NO: 43 - DNA, 60 bp, Artificial sequence
  Synthetic
gcggccgcttacaccgacatcgaaatgaaccgcctcggcaagtagatgagcgccgttttc

SEQ ID NO: 44 - DNA, 39 bp, Artificial sequence
  Synthetic
tcgagctcgagcccggggatccgcgctcggatagggacc

(19) United States
(12) Patent Application Publication
(10) Pub. No.: US 2024/0026443 A1
(43) Pub. Date: Jan. 25, 2024

(54) USE OF A DOUBLE-STRANDED DNA CYTOSINE DEAMINASE FOR MAPPING DNA-PROTEIN INTERACTIONS

(71) Applicant: University of Washington, Seattle, WA (US)

(72) Inventors: Joseph D. Mougous, Seattle, WA (US); Marcos H. de Moraes, Seattle, WA (US); Snow Brook Peterson, Seattle, WA (US)

(73) Assignee: University of Washington, Seattle, WA (US)

(21) Appl. No.: 18/246,981

(22) PCT Filed: Sep. 29, 2021
(86) PCT No.: PCT/US21/52504

§ 371 Date: Mar. 28, 2023

Related U.S. Application Data
(60) Provisional application No. 63/084,829, filed on Sep. 29, 2020.

(51) Int. Cl. C12Q 1/6874; C12Q 1/6804

(52) U.S. Cl. CPC C12Q 1/6874; C12Q 1/6804

(57) ABSTRACT

The disclosure provides methods and related compositions and kits for mapping DNA-protein interactions (DPIs). In one aspect, the disclosed methods comprise contacting a double stranded DNA molecule with a target protein; coupling a double stranded DNA deaminase (DddA) to the target protein, before or after the contacting step; permitting deamination of one or more cytosine residues in a domain of the double stranded DNA molecule by the DddA to provide one or more uracil residues, wherein the domain comprises a site of interaction between the target protein and the double stranded DNA molecule; determining the sequence of at least a portion of the double stranded DNA molecule; and detecting the domain comprising one or more cytosine deamination events. The method can be controlled by use of DddA inhibitors. The method can also incorporate use of inhibiting a base-excision repair pathway when addressing DPIs in a cellular context.

### CROSS-REFERENCE TO RELATED APPLICATION

This application claims the benefit of Provisional Application No. 63/084,829, filed Sep. 29, 2020, the disclosure of which is incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 3915-P1152WOUW_Seq_List_FINAL_20210928_ST25.txt. The text file is 19 KB; was created on Sep. 28, 2021; and is being submitted via EFS-Web with the filing of the specification.

### BACKGROUND

A wide variety of proteins interact with DNA to facilitate myriad cellular functions, including control and regulation of gene expression. Many technologies have been developed to facilitate analysis of where and how a protein of interest interacts with DNA. Advances in DNA sequencing have promoted rapid expansion in DNA-protein interaction (DPI) mapping technologies and their applications. Chromatin immunoprecipation sequencing (ChIP-seq) became an early standard for studying both prokaryotic and eukaryotic systems. In this approach, DPIs are identified through chemical crosslinking of DNA-protein complexes, DNA fragmentation, immunoprecipitation of a DNA binding protein (DBP) of interest, crosslink reversal, DNA purification, and DNA sequencing. Sample preparation is technically challenging and requires approximately one week to implement. More recently, Cut&Run and related technologies have gained popularity as alternatives to ChIP-seq. These techniques offer several advantages relative to ChIP-seq including low starting material quantities that permit single cell measurements, the absence of crosslinking and its associated artifacts, and reduced sequencing with improved signal-to-noise.

Although powerful, ChIP-seq and Cut&Run-related approaches are fundamentally ex vivo technologies and cannot capture DPIs in living cells. A method that overcomes this limitation is DNA adenine methyltransferase identification (DamID), where the DBP of interest is fused to DAM and DPI site identification occurs through restriction enzyme or antibody mediated methylation site enrichment. However, the utility of this technique is limited by low resolution (1 kb) owing to the frequency of DAM recognition sites (GATC) and by toxicity resulting from widespread adenine methylation. A second approach that facilitates the mapping of DPIs in vivo employs mapping the sites of insertion of so-called self-reporting transposons (SRTs). In this technique, a transposase is fused to the DBP of interest, and DPIs are identified by DNA or RNA sequencing to determine sites of transposon insertion. A major limitation to this approach is that transposon insertions occur at low frequency within individual cells (15-100 events per cell), and thus the technology it is not amenable to single cell studies. Additionally, the accumulation of transposon insertions within a population may cause phenotypic consequences through gene disruption.

Notwithstanding the improvements to ChIP sequencing and other methods of mapping DNA-protein interactions (DPIs), a need remains for simple and efficient strategies to determine precise location of protein interactions on DNA. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of mapping one or more DNA-protein interactions (DPIs). The method comprises
(a) contacting a double stranded DNA molecule with a target protein;
(b) coupling a double stranded DNA deaminase (DddA) to the target protein;
(c) permitting deamination of one or more cytosine residues in a domain of the double stranded DNA molecule by the DddA to provide one or more uracil residues within the domain, wherein the domain comprises a DPI site for the target protein and the double stranded DNA molecule;
(d) determining the sequence of at least a portion of the double stranded DNA molecule; and
(e) detecting the domain comprising one or more cytosine deamination events, thereby mapping the DPI site for the target protein and the double stranded DNA molecule.

In some embodiments, the coupling of the DddA to the target protein occurs before the contacting of step (a). In some embodiments, the coupling of the DddA to the target protein occurs after the contacting of step (a).

In some embodiments, the double stranded DNA molecule is genomic DNA in a cell.

In some embodiments, the DddA comprises a DddA domain with an amino acid sequence with at least about 85% identity to SEQ ID NO:1. In some embodiments, the coupling of step (b) comprises providing a fusion protein comprising a target protein domain and a DddA domain, optionally wherein the DddA domain comprises an amino acid sequence with at least about 85% identity to SEQ ID NO:1. In some embodiments, the fusion protein further comprising a linker domain disposed between the target protein domain and the DddA domain. In some embodiments, the fusion protein comprises an amino acid sequence with at least about 85% sequence identity to SEQ ID NO:7. In some embodiments, the double stranded DNA molecule is genomic DNA in a cell that further comprises a nucleic acid encoding the fusion protein, and wherein the contacting of step (a) comprises permitting expression of the fusion protein from the nucleic acid.

In some embodiments, the DddA is indirectly coupled to the target protein. In some embodiments, the DddA is coupled to an affinity reagent that specifically binds to the target protein. In some embodiments, the double stranded DNA molecule is genomic DNA in a cell and the coupling of step (b) comprises contacting the cell with the DddA coupled to the affinity reagent and permitting the affinity reagent to specifically bind to the target protein. In some embodiments, the method further comprises permeabilizing the cell.

In some embodiments, the method further comprises providing a DddA inhibitor, wherein the permitting deamination step (c) comprises removing, or depleting levels of, the DddA inhibitor. In some embodiments, the DddA inhibitor is a double stranded DNA deaminase A immunity (DddAI) protein. In some embodiments, the DddA inhibitor comprises an amino acid sequence with at least about 85% to SEQ ID NO:2. In some embodiments, the double stranded DNA molecule is genomic DNA in a cell and the coupling of step (b) comprises expressing a fusion protein comprising a target protein domain and DddA domain in the cell, and wherein providing the DddA inhibitor comprises transiently expressing the DddAI protein in the cell.

In some embodiments, the double stranded DNA molecule is genomic DNA in a cell and the method further comprises inhibiting a base-excision repair pathway in the cell. In some embodiments, inhibiting the base-excision repair pathway in the cell comprises introducing a genetic modification to the cell to reduce or prevent expression of functional uracil DNA glycosylase (UNG) in the cell. In some embodiments, inhibiting the base-excision repair pathway in the cell comprises providing the cell with an UNG inhibitor. In some embodiments, providing the cell with an UNG inhibitor comprises contacting the cell with the UNG inhibitor. In some embodiments, providing the cell with an UNG inhibitor comprises expressing the UNG inhibitor in the cell. In some embodiments, the UNG inhibitor is uracil glycosylase inhibitor protein (Ugi). In some embodiments, the UNG inhibitor comprises an amino acid sequence with at least about 85% sequence identity to SEQ ID NO:3.

In some embodiments, the target protein directly interacts with the double stranded DNA molecule. In some embodiments, the target protein indirectly interacts with the double stranded DNA molecule through one or more intervening proteins. In some embodiments, target protein is a putative transcription factor.

In some embodiments, the one or more cytosine deamination events in step (e) comprises detecting an accumulation of one or more C to T mutations in the domain. In some embodiments, detecting the accumulation of one or more C to T mutations in the domain comprising comparing the determined sequence with the sequence of a reference DNA molecule that was not contacted with a DddA.

In some embodiments, the double stranded DNA molecule is genomic DNA in a cell, and wherein the cell is a prokaryotic cell or eukaryotic cell. In some embodiments, the eukaryotic cell is a fungal cell, plant cell, or animal cell, such as insect cell, mammalian cell, and the like.

In another aspect, the disclosure provides a fusion protein comprising a DNA deaminase (DddA) domain and a target protein domain.

In some embodiments, the DddA domain comprises an amino acid sequence with at least about 85% identity to SEQ ID NO:1. In some embodiments, the method further comprises a linker domain disposed between the target protein domain and the DddA domain. In some embodiments, the fusion protein comprises an amino acid sequence with at least about 85% sequence identity to SEQ ID NO:7.

In another aspect, the disclosure provides a nucleic acid encoding the fusion protein as described herein.

In another aspect, the disclosure provides a vector comprising the nucleic acid as described herein, further comprising an expression promoter sequence operatively linked to the nucleic acid encoding the fusion protein.

In another aspect, the disclosure provides a kit comprising one of: a target protein and a DNA deaminase (DddA), optionally wherein the target protein and DddA are coupled, or optionally wherein the target protein and the DddA are separate and wherein the DddA is linked to an affinity reagent that specifically binds to the target protein; the fusion protein as described herein; or the vector as described herein.

In some embodiments, the kit further comprises one or more of: a DddA inhibitor or a vector encoding the DddA inhibitor; a uracil DNA glycosylase (UNG) inhibitor or a vector encoding the UNG inhibitor; and a cell permeabilizing agent.

In some embodiments, the DddA inhibitor comprises an amino acid sequence with at least about 85% to SEQ ID NO:2. In some embodiments, the UNG inhibitor comprises an amino acid sequence with at least about 85% to SEQ ID NO:3.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1G illustrate that 3D-seq can accomplish DPI mapping in vivo as illustrated in studies of P. aeruginosa GcsR. (TA) Diagram providing an overview of the 3D-seq method. Top, cell schematic containing the genetic elements required for 3D-seq. Elements may be integrated into the chromosome or supplied on plasmids. Middle, model depicting localized activity of DddA (10) when fused to a DBP of interest (20) and after growth in the absence of arabinose to limit production of DddAI (30). Bottom, schematized 3D-seq output indicating enrichment of C⋅G-to-T⋅A transitions (40) in the vicinity of a DPI site (50). (1B) Growth yield (normalized to wild-type) of the indicated strains on minimal medium containing glycine or succinate as the sole carbon source. (1C-1F) Average (n=4) C⋅G-to-T⋅A transition frequency by genome position after passaging cultures of P. aeruginosa bearing the indicated genotypes, in the presence or absence of arabinose (Ara) to induce DddAI expression. Data were filtered to remove a prophage hypervariable region and positions with low sequence coverage (<15-fold read depth), and positions with an average transition frequency<0.004 were removed ease of visualization. (1G) Zoomed view of a subset of the data depicted in (f). Location of the previously characterized GcsR binding site (60) and adjacent genetic elements shown to scale above.

FIGS. 2A-2D graphically illustrate that statistical analyses and data filtering enhance signal-to-noise and allow 3D-seq to precisely map DPIs. (2A and 2B) Average (n=4) C⋅G-to-T⋅A transition frequency within the (2A) primary GcsR 3D-seq peak region or (2B) a control region located 100,000 bp upstream, with positions colored by the number of replicates in which a transition at that position was observed. (2C) Moving average (75 bp window) of C⋅G-to-T⋅A transition frequencies and the curve deriving from the statistical model (line) calculated from filtered 3D-seq data for the GcsR peak region (see methods). Y-coordinates for the model curve are scaled arbitrarily. (2D) Genome-wide moving average (75 bp window) of C⋅G-to-T⋅A transition frequencies calculated for GcsR 3D-seq data after filtering as in (2C).

FIGS. 3A-3H graphically illustrate that 3D-seq maps DPIs for P. aeruginosa transcription factors belonging to different families and with varying numbers of binding sites. (3A-3H) Graphically illustrate the moving average (n=4, 75 bp window) of C⋅G-to-T⋅A transition frequencies calculated from filtered 3D-seq data deriving from the indicated P. aeruginosa strains expressing GacA-DddA (3A-3D) or FleQ-DddA (3E-3H) grown with 0.0005% w/v arabinose for induction of DddAI-F. Genome-wide (3A, 3D, 3E) and zoomed (3B, 3C, 3F-3H) regions of the data shown in (3A) or (3E) are provided. Curves deriving from the statistical model (line) calculated from filtered 3D-seq data are shown in the zoomed regions. Y-coordinates for the model curves are scaled arbitrarily. Points in 3F are psIA, points in 3G are PA2869, and points in 3H are cdrA.

FIGS. 4A-4D graphically illustrate that transition mutations associated with GcsR:DddA activity accumulate over time. (4A-4D) Average (n=4) C⋅G-to-T⋅A transition frequency within the primary GcsR 3D-seq peak region after the indicated growth period and in the absence of arabinose. Data were filtered as in FIGS. 1A-1G. The arrow indicates the approximate position of the known GcsR binding site.

FIGS. 5A and 5B graphically illustrate that Ugi expression can substitute for genetic inactivation of ung in 3D-seq. (5A and 5B) Moving average (n=4, 75 bp window) of C⋅G-to-T⋅A transition frequencies calculated from filtered 3D-seq data deriving from the indicated P. aeruginosa strains grown in the absence of arabinose for 20 hrs. IPTG was included to induce the expression of Ugi throughout the growth period. The location of the previously characterized GcsR binding site (70) and adjacent genetic elements are shown to scale above.

FIG. 6 spatially illustrates that the C-terminus of DddAI abuts DddA. The figures provides X-ray crystal structure of the DddAI-DddA complex in ribbon and surface representation, respectively. The C-terminal amino acid of DddAI (Leu123) is indicated by (80) and is shown in space filling representation to highlight its position against the surface of DddA.

### DETAILED DESCRIPTION

DNA-protein interactions (DPIs) are central to fundamental cellular processes such as transcription, chromosome maintenance, and chromosome organization. The spatiotemporal dynamics of these interactions dictate their functional consequences; therefore, there is great interest in facile methods for defining the sites of DPI within cells. The disclosure is based on the inventors' development of a method platform for mapping DPI sites in vivo using the double stranded DNA-specific cytosine deaminase toxin DddA. As described in more detail below, the platform, referred to as DddA-sequencing (3D-seq), leverages the functionality of DddA to deaminate cytosine residues to uracil residues in double stranded DNA and allows controlled implementation of detectable deamination events within a limited region or domain containing an interaction event between a target protein of interest and the DNA. In the illustrated embodiments, the platform entails generating a translational fusion of DddA to a DNA binding protein of interest, inactivating uracil DNA glycosylase, modulating DddA activity via its natural inhibitor protein, and DNA sequencing for genome-wide DPI detection. The method was successfully applied to three Pseudomonas aeruginosa transcription factors that represent divergent protein families and bind variable numbers of chromosomal locations. 3D-seq offers several advantages over existing technologies including ease of implementation and the possibility to measure DPIs at single-cell resolution.

Accordingly, in one aspect the disclosure provides a method of mapping one or more DNA-protein interactions (DPIs). The method comprises:
(a) contacting a double stranded DNA (dsDNA) molecule with a target protein,
(b) coupling a double stranded DNA deaminase (DddA) to the target protein;
(c) permitting deamination of one or more cytosine residues in a domain of the double stranded DNA molecule by the DddA to provide one or more uracil residues within the domain, wherein the domain comprises a DPI site for the target protein and the double stranded DNA molecule;
(d) determining the sequence of at least a portion of the double stranded DNA molecule; and
(e) detecting the domain comprising one or more cytosine deamination events, thereby mapping the DPI site for the target protein and the double stranded DNA molecule.

As used herein, the term “mapping” refers to the observance of a site of interest, e.g., a DNA-protein interaction (DPI) site for the desired target protein and the dsDNA, on a DNA molecule and/or determination or estimation of its relative location on the DNA molecule. The present method can be applied in a variety of contexts and can predict site of interest (e.g., the DPI) with varying resolutions, for example, as distant as 500 bp and as close as 15 bp.

The disclosed method is particularly useful for querying sites of DPI in genomic DNA, including in living cells, although the disclosure also encompasses embodiments where the dsDNA is in a preserved cell, in a cell lysate, or other appropriate reaction mixture. For ease of illustration, the disclosure mostly addresses embodiments involving genomic DNA in a living cell. The method can be performed at the single-cell level, or can be scaled up to be performed in a plurality of cells in independent, parallel assays, or can be performed in bulk in a plurality of cells. The disclosure is not limited to any type of cells, but instead can be broadly applied to any cell-type of interest. For example, the cell can be prokaryotic or eukaryotic, e.g., fungal cell, plant cell, animal cell, e.g., insect cell, mammalian cell, and the like.

The method generally relies on selectively targeting a protein or protein fragment with deaminase activity to site(s) on dsDNA corresponding to DPI(s) such that the limited region(s) around (i.e., proximal in the upstream and downstream directions) to the DPI site(s) is/are uniquely subjected to the deaminase activity. The deaminase activity can then be detected. In some embodiments, the deaminase activity is detected by subsequent sequence analysis where C⋅G-to-T⋅A transitions are noted in the sequence, e.g., relative to a reference sequence. If the DNA template with deamination event is not replicated before analysis, then the uracils (i.e., deaminated cytosines) can be read as thymines.

As used herein, “DNA deaminase (DddA)” refers to an enzyme, or a functional fragment or domain thereof, that deaminates nucleotide residues in double stranded DNA (dsDNA). In some embodiments, the DddA has cytosine deaminase capability. For example, as described below, assays incorporated functional domains of DddA, which is a bacterial toxin-derived cytosine deaminase. In some embodiments, the DddA comprises a deaminase domain with the amino acid sequence set forth in SEQ ID NO:1, or an amino acid sequence with at least 85% identity thereto, for example about 85%, 86%, 87%, 88%, 89%, 90%, 91%, 92%, 93%, 94%, 95%, 96%, 97%, 98%, or 99% sequence identity to SEQ ID NO: 1, or a functional fragment thereof. The functionality of any fragment can be readily determined by a simple confirmatory assay that includes exposing the selected fragment to dsDNA and observing whether nucleotides are deaminated. This determination can be inferred, e.g., in living cells, by permitting the replication of the DNA with deaminated residues and noting presence of C⋅G-to-T⋅A transitions.

The DddA component of the method is targeted to a specific location along the dsDNA by a target protein, which is contacted to the dsDNA. The target protein is not limited and can be any protein, protein fragment, or protein domain that interacts, directly or indirectly, with the dsDNA. For example, the target protein can interact directly with dsDNA by binding to the dsDNA, possibly in a sequence specific manner. Examples include transcription factors. In other embodiments, the target protein can indirectly interact with the dsDNA by association with one or more intervening proteins or molecules that interact with the dsDNA. For example, the intervening proteins or molecules can be transcription factors, histone proteins, proteins that interact with or modify histones or DNA.

To illustrate, exemplary transcription factors that serve as (or providing domains that serve as) the target protein or intervening protein include but are not limited to AAF, abl, ADA2, ADA-NF1, AF-1, AFP1, AhR, AIIN3, ALL-1, alpha-CBF, alpha-CP 1, alpha-CP2a, alpha-CP2b, alphaHo, alphaH2-alphaH3, Alx-4, aMEF-2, AML1, AMLla, AMLlb, AMLlc, AMLlDeltaN, AML2, AML3, AML3a, AML3b, AMY-1L, A-Myb, ANF, AP-1, AP-2alphaA, AP-2alphaB, AP-2beta, AP-2gamma, AP-3 (1), AP-3 (2), AP-4, AP-5, APC, AR, AREB6, Art, Amt (774 M form), ARP-1, ATBF1-A, ATBF1-B, ATF, ATF-1, ATF-2, ATF-3, ATF-3deltaZIP, ATF-a, ATF-adelta, ATPF1, Barhll, Barhl2, Barxl, Barx2, Bcl-3, BCL-6, BD73, beta-catenin, Binl, B-Myb, BP1, BP2, brahma, BRCA1, Brn-3a, Brn-3b, Brn-4, BTEB, BTEB2, B-TFIID, C/EBPalpha, C/EBPbeta, C/EBPdelta, CACC binding factor, Cart-1, CBF (4), CBF (5), CBP, CCAAT-binding factor, CCMT-binding factor, CCF, CCG1, CCK-la, CCK-lb, CD28RC, cdk2, cdk9, Cdx-1, CDX2, Cdx-4, CFF, ChxlO, CLIMI, CLIM2, CNBP, CoS, COUP, CPl, CPIA, CPIC, CP2, CPBP, CPE binding protein, CREB, CREB-2, CRE-BP1, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin Tl, cyclin T2, cyclin T2a, cyclin T2b, DAP, DAX1, DB1, DBF4, DBP, DbpA, DbpAv, DbpB, DDB, DDB-1, DDB-2, DEF, deltaCREB, deltaMax, DF-1, DF-2, DF-3, Dlx-1, Dlx-2, Dlx-3, DIx4 (long isoform), Dlx-4 (short isoform, Dlx-5, Dlx-6, DP-1, DP-2, DSIF, DSIF-pl4, DSIF-pl60, DTF, DUX1, DUX2, DUX3, DUX4, E, El 2, E2F, E2F+E4, E2F+pl07, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EIIaE-A, EIIaE-B, EIIaE-Calpha, EIIaE-Cbeta, EivF, Elf-i, Elk-1, Emx-1, Emx-2, Emx-2, En-1, En-2, ENH-bind. prot, ENKTF-1, EPASl, epsilonFl, ER, Erg-1, Erg-2, ERR1, ERR2, ETF, Ets-1, Ets-1 deltaVil, Ets-2, Evx-1, F2F, factor 2, Factor name, FBP, f-EBP, FKBP59, FKHL18, FKHRL1P2, Fli-1, Fos, FOXB1, FOXC1, FOXC2, FOXD1, FOXD2, FOXD3, FOXD4, FOXE1, FOXE3, FOXF1, FOXF2, FOXGla, FOXGlb, FOXGlc, FOXH1, FOXI1, FOXJla, FOXJlb, FOXJ2 (long isoform), FOXJ2 (short isoform), FOXJ3, FOXKla, FOXKlb, FOXKlc, FOXL1, FOXMla, FOXMlb, FOXMlc, FOXN1, FOXN2, FOXN3, FOXOla, FOXOlb, FOX02, FOX03a, FOX03b, FOX04, FOXP1, FOXP3, Fra-1, Fra-2, FTF, FTS, G factor, G6 factor, GABP, GABP-alpha, GABP-betal, GABP-beta2, GADD 153, GAF, gammaCMT, gammaCACl, gammaCAC2, GATA-1, GATA-2, GATA-3, GATA-4, GATA-5, GATA-6, Gbx-1, Gbx-2, GCF, GCMa, GCNS, GF1, GLI, GLI3, GR alpha, GR beta, GRF-1, Gsc, Gscl, GT-IC, GT-IIA, GT-IIBalpha, GT-IIBbeta, H1TF1, H1TF2, H2RIIBP, H4TF-1, H4TF-2, HAND1, HAND2, HB9, HDAC1, HDAC2, HDAC3, hDaxx, heat-induced factor, HEB, HEBl-p67, HEBl-p94, HEF-1 B, HEF-1T, HEF-4C, HEN1, HEN2, Hesxl, Hex, HIF-1, HIF-lalpha, HIF-lbeta, HiNF-A, HiNF-B, HINF-C, HINF-D, HiNF-D3, HiNF-E, HiNF-P, HIP1, HIV-EP2, Hlf, HLTF, HLTF (Metl23), HLX, HMBP, HMG I, HMG I(Y), HMG Y, HMGI-C, HNF-IA, HNF-IB, HNF-IC, HNF-3, HNF-3alpha, HNF-3beta, HNF-3gamma, HNF4, HNF-4alpha, HNF4alphal, HNF-4alpha2, HNF-4alpha3, HNF-4alpha4, HNF4gamma, HNF-6alpha, hnRNP K, HOX11, HOXAl, HOXAIO, HOXAIO PL2, HOXA11, HOXA13, HOXA2, HOXA3, HOXA4, HOXA5, HOXA6, HOXA7, HOXA9A, HOXA9B, HOXB-1, HOXB13, HOXB2, HOXB3, HOXB4, HOXBS, HOXB6, HOXA5, HOXB7, HOXB8, HOXB9, HOXC10, HOXC11, HOXC12, HOXC13, HOXC4, HOXC5, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Idl, Idl H′, Id2, Id3, Id3/Heir-1, IF1, IgPE-1, IgPE-2, IgPE-3, IkappaB, IkappaB-alpha, IkappaB-beta, IkappaBR, II-1 RF, IL-6 RE-BP, 11-6 RF, INSAF, IPF1, IRF-1, IRF-2, B, IRX2a, Irx-3, Irx-4, ISGF-1, ISGF-3, ISGF3alpha, ISGF-3gamma, 1st-1, ITF, ITF-1, ITF-2, JRF, Jun, JunB, JunD, kappay factor, KBP-1, KER1, KER-1, Koxl, KRF-1, Ku autoantigen, KUP, LBP-1, LBP-la, LBXl, LCR-Fl, LEF-1, LEF-IB, LF-A1, LHX1, LHX2, LHX3a, LHX3b, LHXS, LHX6.1a, LHX6.1b, LIT-1, Lmol, Lmo2, LMX1A, LMX1B, L-Myl (long form), L-Myl (short form), L-My2, LSF, LXRalpha, LyF-1, Lyl-1, M factor, Madl, MASH-1, Maxl, Max2, MAZ, MAZ1, MB67, MBF1, MBF2, MBF3, MBP-1 (1), MBP-1 (2), MBP-2, MDBP, MEF-2, MEF-2B, MEF-2C (433 AA form), MEF-2C (465 AA form), MEF-2C (473 M form), MEF-2C/delta32 (441 AA form), MEF-2D00, MEF-2D0B, MEF-2DA0, MEF-2DAO, MEF-2DAB, MEF-2DA′B, Meis-1, Meis-2a, Meis-2b, Meis-2c, Meis-2d, Meis-2e, Meis3, Meoxl, Meoxla, Meox2, MHox (K-2), Mi, MIF-1, Miz-1, MM-1, MOP3, MR, Msx-1, Msx-2, MTB-Zf, MTF-1, mtTFl, Mxil, Myb, Myc, Myc 1, Myf-3, Myf-4, Myf-5, Myf-6, MyoD, MZF-1, NCI, NC2, NCX, NELF, NERI, Net, NF Ill-a, NF NF-1, NF-IA, NF-1B, NF-1X, NF-4FA, NF-4FB, NF-4FC, NF-A, NF-AB, NFAT-1, NF-AT3, NF-Atc, NF-Atp, NF-Atx, Nf etaA, NF-CLEOa, NF-CLEOb, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NF-kappaB(-like), NF-kappaBl, NF-kappaB 1, precursor, NF-kappaB2, NF-kappaB2 (p49), NF-kappaB2 precursor, NF-kappaEl, NF-kappaE2, NF-kappaE3, NF-MHCIIA, NF-MHCIIB, NF-muEl, NF-muE2, NF-muE3, NF-S, NF-X, NF-X1, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A vl, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-5b, NP-TCII, NR2E3, NR4A2, Nrfl, Nrf-1, Nrf2, NRF-2betal, NRF-2gammal, NRL, NRSF form 1, NRSF form 2, NTF, 02, OCA-B, Oct-1, Oct-2, Oct-2.1, Oct-2B, Oct-2C, Oct-4A, Oct4B, Oct-5, Oct-6, Octa-factor, octamer-binding factor, oct-B2, oct-B3, Otxl, Otx2, OZF, pl07, pl30, p28 modulator, p300, p38erg, p45, p49erg,-p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx-la, Pbx-lb, Pbx-2, Pbx-3a, Pbx-3b, PC2, PC4, PC5, PEA3, PEBP2alpha, PEBP2beta, Pit-1, PITX1, PITX2, PITX3, PKNOX1, PLZF, PO-B, Pontin52, PPARalpha, PPARbeta, PPARgammal, PPARgamma2, PPUR, PR, PR A, pRb, PRD1-BF1, PRDI-BFc, Prop-1, PSE1, P-TEFb, PTF, PTFalpha, PTFbeta, PTFdelta, PTFgamma, Pu box binding factor, Pu box binding factor (B JA-B), PU.1, PuF, Pur factor, Rl, R2, RAR-alphal, RAR-beta, RAR-beta2, RAR-gamma, RAR-gammal, RBP60, RBP-Jkappa, Rel, RelA, RelB, RFX, RFX1, RFX2, RFX3, RFXS, RF-Y, RORalphal, RORalpha2, RORalpha3, RORbeta, RORgamma, Rox, RPF1, RPGalpha, RREB-1, RSRFC4, RSRFC9, RVF, RXR-alpha, RXR-beta, SAP-la, SAPlb, SF-1, SHOX2a, SHOX2b, SHOXa, SHOXb, SHP, SIII-pl lO, SIII-pl5, SIII-pl8, SIM′, Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Spl, Sp2, Sp3, Sp4, Sph factor, Spi-B, SPIN, SRCAP, SREBP-la, SREBP-lb, SREBP-lc, SREBP-2, SRE-ZBP, SRF, SRY, SRPl, Staf-50, STATlalpha, STATlbeta, STAT2, STAT3, STAT4, STAT6, T3R, T3R-alphal, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1, Tal-lbeta, Tal-2, TAR factor, TBP, TBX1A, TBX1B, TBX2, TBX4, TBXS (long isoform), TBXS (short isoform), TCF, TCF-1, TCF-IA, TCF-1B, TCF-1C, TCF-1D, TCF-IE, TCF-1F, TCF-1G, TCF-2alpha, TCF-3, TCF-4, TCF-4(K), TCF-4B, TCF-4E, TCFbetal, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MAT1, TFIIH-M015, TFIIH-p34, TFIIH-p44, TFIIH-p62, TFIIH-p80, TFIIH-p90, TFII-I, Tf-LFl, Tf-LF2, TGIF, TGIF2, TGT3, THRA1, TIF2, TLE1, TLX3, TMF, TR2, TR2-11, TR2-9, TR3, TR4, TRAP, TREB-1, TREB-2, TREB-3, TREFl, TREF2, TRF (2), TTF-1, TXRE BP, TxREF, UBF, UBP-1, UEF-1, UEF-2, UEF-3, UEF-4, USF1, USF2, USF2b, Vav, Vax-2, VDR, vHNF-1A, vHNF-1B, vHNF-1C, VITF, WSTF, WT1, WT1I, WT1 I-KTS, WT1 I-del2, WT1-KTS, WTl-del2, X2BP, XBP-1, XW-V, XX, YAF2, YB-1, YEBP, YY1, ZEB, ZF1, ZF2, ZFX, ZHX1, ZIC2, ZID, ZNF 174, amongst others. Persons skilled in the art can readily select other proteins that interact directly with DNA, are components of the chromatin, and/or otherwise interact with chromatin or other elements of transcriptional machinery (i.e., interact indirectly with DNA).

As indicted above, the DddA is coupled, before, during, or after the contacting of step (a) to the target protein to facilitate the targeting. The coupling can incorporate a covalent bond or non-covalent interactions. The coupling can be direct between the DddA and the target protein, or indirect where there is an intervening amino acid sequence, or molecule (or molecules).

For example, in some embodiments, the coupling can occur before the contacting of step (a). In some embodiments, the coupling of step (b) comprises providing a fusion protein with at least a target protein domain and a DddA domain. The target protein domain is the target protein in step (a), as described above, and the DddA domain is the DddA in step (b), as described above. The fusion protein can comprise the target protein domain and the DddA domain in any order from the N terminus to the C terminus. In some embodiments, the target protein domain and the DddA domain are separated by a linker. A linker is typically a stretch of amino acid residues that has no functional (e.g., enzymatic) role, but instead provides separation and flexibility between the target protein domain and a DddA domain to allow each to perform their respective functions without steric hindrance between them. Many linker and conjugation technologies are known and are encompassed by this disclosure. The length of the linker is not critical and is preferably of a length that avoids or decreases steric hindrance between the DddA domain and the target protein domain. Thus, the linker can be a peptide with at least a single amino acid, such as 2, 3, 4, 5, 6, 7, 8, 9, 10 or more amino acids. However, it will be understood that the linker can be substantially longer, ranging from 10 to 15, 15 to 25, 25 to 50, 50 to 100, or any range contained therein, or even more amino acids long. The linker can be flexible to facilitate activity of each domain in the fusion protein. Furthermore, in some embodiments, the linker domain is not reactive. For example, the linker domain does not substantially interact with cytosolic components. In some embodiments, the linker can comprise one or more alanine residues, serine residues, glycine residues, or a combination thereof. In one illustrative, non-limiting embodiment, the linker has a sequence with at least 80% sequence identity, e.g., 85%, 90%, 95%, 98%, or 100% sequence identity, to the amino acid sequence set forth in SEQ ID NO:6, which was used to develop initial embodiments of the method.

For illustration purposes, in some embodiments, the fusion protein comprises a linker domain with at least 80% sequence identity to SEQ ID NO: 6, disposed between a DddA domain with at least about 85% identity to SEQ ID NO: 1, as described above, and a target domain. An illustrative, non-limiting target domain is GcsR, a sigma 54-dependent transcription activator of an operon encoding the glycine cleavage system, which was used in the experiments described in Example 1 to target the DddA to DPI sites in a cell's genome. A non-limiting example of a fusion protein sequence, including a target protein domain (i.e., GcsR), a linker domain, and a DddA domain is set forth in SEQ ID NO:7.

In contexts where the dsDNA is genomic DNA in a cell, the step of contacting the dsDNA can be accomplished by expressing the fusion protein comprising the DddA and the target protein in the same cell. This transgenic expression of the fusion protein, described in more detail above, can be implemented using any known vector or expression system available for the cell-type of interest without limitation. Simple stated. Exogenous nucleic acid encoding the fusion protein, e.g., in an expression vector system, can be introduced into the cell and conditions for expression can be provided. The fusion protein can be introduced in a manner that provides transient or inducible expression or constitutive expression. The exogenous nucleic acid can be integrated in the genome of the cell or can remain on an expression construct separate from the genome, as can be implemented by persons skilled in the art using appropriate vectors. With such transgenic gene design, the contacting of step (a) comprises permitting or inducing expression of the fusion protein such that it will contact the genomic DNA in the cell.

Exemplary embodiments of non-covalent, direct coupling of the DddA to the target protein include associations between biotin and avidin/streptavidin, which are attached respectively to each protein component, according to known techniques.

In alternative embodiments, the DddA is indirectly coupled to the target protein. Indirect coupling encompasses any non-covalent binding between the target protein and the DddA. For example, in some embodiments the DddA is linked to an affinity reagent that specifically binds to the target protein. Other embodiments include one or more additional intervening affinity reagents. For example, the DddA can be linked to a first affinity reagent, which specifically binds a second affinity reagent, which specifically binds to the target protein. A person skilled in the art will understand that the indirect coupling can include yet further affinity reagents (i.e., a third affinity reagent, etc.) that intervene in the indirect coupling between the DddA and the target protein. Thus, in some embodiments, the double stranded DNA molecule is genomic DNA in a cell and the method further comprises contacting the cell with the DddA linked to the affinity reagent in a complex, and permitting the affinity reagent to specifically bind to the target protein or an intervening affinity reagent that is, in turn, associated with the target protein. The affinity reagents can be or comprise antibodies, antibody-like molecules, DARPins, aptamer, and other antigen binding molecules, which can be readily generated accordingly to skill in the art to selectively bind to a target antigen of choice. Additional description is provided below.

In some embodiments, the cell can be permeabilized prior to the start of the method, or implementation of the method further comprises permeabilizing the cell, to facilitate delivery of the target protein and/or the DddA, as independent components or linked in a complex or fusion protein, as described above. The cell can be permeabilized by contacting the cell and/or nucleus with a permeabilizing agent, such as with a detergent, for example Triton and/or NP-40 or another agent, such as digitonin. Other appropriate permeabilizing agents can be readily selected by persons skilled in the art. To illustrate, in a specific non-limiting embodiment the method comprises permeabilizing the cell, contacting the cell with the target protein, permitting the target protein to bind to the cell's genomic DNA, and then contacting the cell with the DddA and permitting the DddA to couple to the target protein (e.g., via an affinity reagent linked to the DddA). Alternatively, the target protein can be expressed in the cell, after which the cell is permeabilized and contacted with the DddA and allowed to couple with the target protein as it is bound to the cell DNA.

As indicated below, on its own DddA can be promiscuous and non-specific in its modifications to dsDNA. Thus, to provide a clearer and accurate signal for mapping DPIs, the off-target activity of DddA can be prevented by controlling when the DddA protein or domain is active. This can be accomplished, for example, by providing a DddA inhibitor, wherein the permitting deamination step (c) comprises removing, or depleting levels of, the DddA inhibitor. In some embodiments, the DddA inhibitor is a double stranded DNA deaminase A immunity (DddI_{A}) protein. An exemplary amino acid sequence for a DddI_{A} protein is provide in SEQ ID NO:2. Thus, in some embodiments, the DddA inhibitor comprises an amino acid sequence with at least about 85%, 86%, 87%, 88%, 89%, 90%, 91%, 92%, 93%, 94%, 95%, 96%, 97%, 98%, or 99% sequence identity to SEQ ID NO:2, or a functional fragment thereof. In some embodiments, the DddA inhibitor is transiently expressed in the cell (e.g., from an exogenously provided expression vector) during the initial contacting steps. Expression of the DddA inhibitor is terminated, inhibited, or otherwise reduced once a sufficient time for the target protein to contact the dsDNA and the DddA is coupled to the target protein. Once the levels of DddA inhibitor are reduced, the DddA is permitted to deaminate one or more cytosine resides in the dsDNA. In an illustrative embodiment, where the double stranded DNA molecule is genomic DNA in a cell, the coupling of step (b) comprises expressing a fusion protein comprising the target protein domain and DddA in the cell. The embodiment further comprises transiently expressing the DddA inhibitor (e.g., DddI_{A} protein) in the cell. Transient expression can be accomplished using techniques familiar in the art. For example, the nucleic acid encoding the DddA inhibitor (e.g., DddI_{A} protein) can be operatively linked to an appropriate promoter that is controllable or inducible. Upon removal of the appropriate factors, the expression of the DddA inhibitor is reduced or stopped, thereby allowing the DddA to have active deaminase activity.

In other embodiments, a limited amount of the DddA inhibitor (e.g., DddI_{A} protein) is contacted to the cell to generally reduce non-specific activity of the DddA. The appropriate levels of DddA inhibitor can be routinely optimized to obtain a detectable signal of DddA deaminase activity that is spatially limited by the association with (i.e., coupling to) the target protein.

In some embodiments where the double stranded DNA molecule is genomic DNA in a cell, the cell has a deficient or negatively modulated base excision repair pathway. In some embodiments, the method further comprises inhibiting a base excision repair pathway in the cell. This inhibition or negative modulation is to prevent the endogenous cellular repair machinery to re-animate the modified cytosines, which would erase the DddA-induced signal. Inhibiting the base-excision repair pathway in the cell can comprise introducing genetic modification to reduce or prevent expression of functional uracil DNA glycosylase (UNG) in the cell. With the target sequence encoding UNG, the genetic modification can be accomplished according to known methods. An exemplary UNG protein is from Pseudomonas aeruginosa and has the amino acid sequence set forth in SEQ ID NO:4. Accordingly, in some embodiments, the target wild-type gene encoding the UNG protein encodes an amino acid sequence with at least about 85%, 86%, 87%, 88%, 89%, 90%, 91%, 92%, 93%. 94%. 95%. 96%, 97%. 98%, or 99% sequence identity to SEQ ID NO:4 prior to the genetic modification. The genetic modification can be an insertion or deletion mutation, a non-conservative substitution mutation, or a missense mutation, that leads to reduced expression of functional protein or lack of any expression of functional protein in the cell. Known techniques to implement the genetic modification of the target base-excision repair pathway gene (e.g., encoding UNG), include use of nucleases to create specific double-stranded break (DSBs) at a desired location in the genome (e.g., the gene encoding UNG), which in some cases harnesses the cells endogenous mechanisms to repair the induced break by natural processes of homologous recombination (HR) and/or nonhomologous end-joining (NHEJ). Genetic modification effectors encompassed in this disclosure include Zinc Finger Nucleases (ZFNs), Transcription Activator-Like Effector Nucleases (TALENs), the Clustered Regularly Interspaced Short Palindromic Repeats/CAS9 (CRISPR/Cas9) system, and meganuclease re-engineered as homing endonucleases.

In other embodiments, inhibiting the base-excision repair pathway in the cell comprises providing the cell with an UNG inhibitor. For example, the inhibitor can be contacted directly to a cell (e.g., a permeabilized cell). In other embodiments the method comprises expressing the UNG inhibitor in the cell, such as, e.g., via expression of a genetically an exogenous transgene introduced into the cell. In some embodiments, the UNG inhibitor is uracil glycosylase inhibitor protein (Ugi). An exemplary Ugi has the amino acid sequence set forth in SEQ ID NO:3. Accordingly, in some embodiments, the expressed or contacted UNG protein inhibitor is encoded by a nucleic acid encoding an amino acid sequence with at least about 85%, 86%, 87%, 88%, 89%, 90%, 91%, 92%, 93%, 94%, 95%, 96%, 97%, 98%, or 99% sequence identity to SEQ ID NO:3. In embodiments where the UNG inhibitor is expressed directly in the cell under study, the nucleic acid can be incorporated into the same vector that delivers the expression cassette encoding the target protein and/or DddA (as described above). In some embodiments, the UNG inhibitor is incorporated into a distinct domain that is fused to the encoded the target protein and/or DddA (as described above). Alternatively, the Ung inhibitor can be expressed from a nucleic acid that has been introduced into the cell via a distinct vector construct.

After the DddA is targeted to a locus of the dsDNA by virtue of coupling to the target protein, the DddA will be anchored to the locus and deaminate one or more cytosine residues within a limited domain that includes the locus. Thus, the domain comprises a site of interaction (direct or indirect) between the dsDNA and the target protein.

Once one or more cytosine residues have been deaminated to uracil residues, the dsDNA is replicated. The replication of the mutated template will incorporate a thymine residue based on the uracil template. Thus, sequencing of the dsDNA after targeted exposure to the DddA according to the method will indicate a cytosine to thymine mutation where a deamination event occurred due to the DddA deaminase activity. Thus, detecting the one or more cytosine deamination events in step (e) comprises detecting an accumulation of one or more C to T mutations in the domain. C to T mutations can be determined by comparison of the determined sequence to a reference sequence. The reference sequence can be derived from a database of known sequences. Alternatively, the reference sequence can be produced in parallel using similar dsDNA that was not contacted with a functional DddA.

In another aspect, the disclosure provides a fusion protein that comprises a DNA deaminase (DddA) domain and a target protein domain. Discussion of the DddA domain and target protein domain are provided above in the context of the method and are also encompassed in this aspect. Briefly, in some embodiments, the DddA domain comprises an amino acid sequence with at least about 85% identity to SEQ ID NO:1. The target protein domain can be any desired protein that directly or indirectly binds to dsDNA.

Examples are described above in more detail. In some embodiments, the fusion protein further comprises a linker domain disposed between the target protein domain and the DddA domain. Linker domains are also described in more detail above. In one example, the linker has an amino acid sequence with at least 80% identity to the SEQ ID NO:6. In some illustrative embodiments, the fusion protein comprises an amino acid sequence with at least about 85% sequence identity to SEQ ID NO:7.

In another aspect, the disclosure provides a nucleic acid molecule encoding any of the fusion proteins described herein. For example, a person of ordinary skill in the art can use the genetic code to determine nucleic acid sequences that can encode fusion proteins comprising a DddA domain and a target protein domain, and optionally a linker domain disposed between the DddA domain and the target protein domain.

In some embodiments, the nucleic acid further comprises a promoter sequence operatively linked to the sequence encoding the fusion protein. The term “promoter” refers to a regulatory nucleotide sequence that can activate transcription (expression) of a gene and/or splice variant isoforms thereof. A promoter is typically located upstream of a gene, but can be located at other regions proximal to the gene, or even within the gene.

The promoter typically contains binding sites for RNA polymerase and one or more transcription factors, which participate in the assembly of the transcriptional complex. As used herein, the term “operatively linked” indicates that the promoter and the encoding nucleic acid are configured and positioned relative to each other a manner such that the promoter can activate transcription of the encoding nucleic acid by the transcriptional machinery of the cell. The promoter can be constitutive or inducible. Constitutive promoters can be determined based on the character of the target cell and the particular transcription factors available in the cytosol. A person of ordinary skill in the art can select an appropriate promoter based on the intended application, as various promoters are known and commonly used in the art.

Accordingly, in other aspects and embodiments, the disclosure provides a vector comprising the nucleic acid described above, and uses thereof to implement the above methods. The vector can be any construct that facilitates the delivery of the nucleic acid to the target cell and/or expression of the nucleic acid within the cell. The vectors can be viral vectors, circular nucleic acid constructs (e.g., plasmids), or nanoparticles.

Various viral vectors are known in the art and are encompassed by the present disclosure. See, e.g., Machida, C. A. (ed.), Viral Vectors for Gene Therapy: Methods and Protocols, Humana Press, Totowa, New Jersey (2003); Muzyczka, N., (ed.), Current Topics in Microbiology and Immunology: Viral Expression Vectors, Springer-Verlag, Berlin, Germany (2012), each incorporated herein by reference in its entirety. In some embodiments, the viral vector is an adeno associated virus (AAV) vector, an adenovirus vector, a retrovirus vector, or a lentivirus vector.

In another aspect, the disclosure provides a cell comprising the nucleic acid encoding any fusion protein, as described herein. In some embodiments, the cell comprises a vector, wherein the vector comprises the nucleic acid encoding any fusion protein as described herein. The cell is capable of expressing the fusion protein from the nucleic acid. For example, the nucleic acid and/or vector comprising the nucleic acid can be configured for expression of the fusion protein from the encoding nucleic acid within the cell. A promoter operatively linked to the nucleic acid can be appropriately configured to allow binding of the cell's RNA polymerase and one or more transcription factors to permit assembly of the transcriptional complex. The disclosure encompasses any type of cell for this aspect.

In yet another aspect, the disclosure provides a kit to facilitate any of the method embodiments described above. The kit comprises reagents for contacting the dsRNA, including embodiments where the reagents facilitate transgenic expression the reagents in a cell to perform steps of the methods. Thus, in some embodiments, the kit comprises at least one of: (a) a target protein and a DNA deaminase (DddA) (e.g., wherein the target protein and DddA are coupled or the target protein and DddA are separate and wherein the DddA is linked to an affinity reagent that specifically binds to the target protein); (b) the fusion protein described above; or (c) the nucleic acid encoding the fusion protein, or a vector comprising the nucleic acid, as described above. The kit can comprise one or more other reagents to facilitate the methods, such as (i) a DddA inhibitor or a vector encoding the DddA inhibitor; (ii) a uracil DNA glycosylase (UNG) inhibitor or a vector encoding the UNG inhibitor; and (iii) a cell permeabilizing agent. Embodiments of these additional reagents are described in more detail above and are encompassed in this aspect. The kit can further comprise additional reagents such as appropriate cell culture media, buffers, tissue culture plates, etc. to facilitate culture of target cells. In some embodiments, the kit further comprises written instructions guiding use of the reagents in the performance of any of the method embodiments described above.

### Additional Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, in some embodiments, the term “about” refers to a number within a range of 10%, 9%, 8%, 7%, 6%, 5%, 4%, 3%, 2%, or 1% above and/or below the indicated reference number.

As used herein, the term “nucleic acid” refers to a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyribo” prefix before the nucleobase to infer the type of five-carbon sugar. For example, “ribocytosine” as occasionally used herein is equivalent to a cytidine residue because it indicates the presence of a ribose sugar in the RNA molecule at that residue. The nucleic acid polymer can be or comprise a deoxyribonucleotide (DNA) polymer, a ribonucleotide (RNA) polymer, including mRNA. The nucleic acids can also be or comprise a PNA polymer, or a combination of any of the polymer types described herein (e.g., contain residues with different sugars) As used herein, the term “polypeptide” or “protein” refers to a polymer in which the monomers are amino acid residues that are joined together through amide bonds.

When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

“Percent sequence identity” or grammatical equivalents means that a particular sequence has at least a certain percentage of nucleic acid or amino acid residues identical to those in a specified reference sequence using an alignment algorithm. Sequence identity and similarity between multiple nucleic acid or polypeptide sequences can be readily determined. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5: 151-3, 1989; Corpet et al, Nuc. Acids Res. 16: 10881-90, 1988; Huang et al. Computer Appls. in the Biosciences 8, 155-65, 1992; and Pearson et al, Meth. Mol. Bio. 24:307-31, 1994. Altschul et al, J. Mol. Biol. 215:403-10, 1990, presents a detailed consideration of sequence alignment methods and homology calculations.

The NCBI Basic Local Alignment Search Tool (BLAST) (Altschul et al, J. Mol. Biol. 215:403-10, 1990) is available from several sources, including the National Center for Biological Information (NCBI, National Library of Medicine, Building 38 A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

The term “wild-type,” “wild-type,” “WT” and the like refers to a naturally-occurring polypeptide or nucleic acid sequence, i.e., one that does not include a man-made variation.

The term “specifically binds” refers to, with respect to a target antigen, the preferential association of an affinity reagent, in whole or part, with a specific antigen, such as a target protein or a transcription factor bound to dsDNA. A specific binding affinity agent binds substantially only to a defined target. It is recognized that a minor degree of non-specific interaction may occur between a molecule, such as a specific affinity reagent, and a non-target antigen. Nevertheless, specific binding can be distinguished as mediated through specific recognition of the antigen. Specific binding typically results in greater than 2-fold, such as greater than 5-fold, greater than 10-fold, or greater than 100-fold increase in amount of bound affinity reagent (per unit time) to a target antigen, such as compared to a non-target antigen. A variety of immunoassay formats are appropriate for selecting affinity reagent specifically reactive with a particular antigen. For example, solid-phase ELISA immunoassays are routinely used to select antibodies specifically immunoreactive with a protein. See Harlow & Lane, Antibodies, A Laboratory Manual, Cold Spring Harbor Publications, New York (1988), for a description of immunoassay formats and conditions that can be used to determine specific reactivity.

In some embodiments, the indicated affinity reagent can be an antibody or an antibody-like molecule.

An “antibody” is a polypeptide ligand that includes at least a light chain or heavy chain immunoglobulin variable region and specifically binds an epitope of an antigen, such as a chromatin associated marker or another affinity reagent. The term “antibody” encompasses antibodies, derived from any antibody-producing mammal (e.g., mouse, rat, rabbit, and primate including human), that specifically bind to an antigen of interest (e.g., a chromatin associated marker or another affinity reagent). Exemplary antibody types include multi-specific antibodies (e.g., bispecific antibodies), humanized antibodies, murine antibodies, chimeric, mouse-human, mouse-primate, primate-human monoclonal antibodies, and anti-idiotype antibodies.

Canonical antibodies can be composed of a heavy and a light chain, each of which has a variable region, termed the variable heavy (VH) region and the variable light (VL) region. Together, the VH region and the VL region are responsible for binding the antigen recognized by the antibody. The term “antibody-like molecule” includes functional fragments of intact antibody molecules, molecules that comprise portions of an antibody, or modified antibody molecules, or derivatives of antibody molecules. Typically, antibody-like molecules retain specific binding functionality, such as by retention of, e.g., with a functional antigen-binding domain of an intact antibody molecule. Preferably antibody fragments include the complementarity-determining regions (CDRs), antigen binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagent does not have to be naturally occurring or naturally derived, but can be further modified to, e.g., reduce the size of the domain or modify affinity for the antigen as necessary. For example, complementarity determining regions (CDRs) can be derived from one source organism and combined with other components of another, such as human, to produce a chimeric molecule that avoids stimulating immune responses in a subject.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Once a monoclonal antibody is identified for inclusion within the bi-specific molecule, the encoding gene for the relevant binding domains can be cloned into an expression vector that also comprises nucleic acids encoding the remaining structure(s) of the bi-specific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phosphodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/target binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

Designed ankyrin repeat proteins (DARPins) are engineered antibody mimetic proteins that can have highly specific and high affinity target antigen binding. DARPins are typically based on natural ankyrin repeat proteins and comprise at least three repeat motifs. Repetitive structural units (motifs) form a stable protein domain with a large potential target interaction surface. Typically, DARPins comprise four or five repeats, of which the first (N-capping repeat) and last (C-capping repeat) serve to shield the hydrophobic protein core from the aqueous environment. DARPins often correspond to the average size of natural ankyrin repeat protein domains. DARPins can be screened and engineered starting from encoding libraries of randomized variations. Once desired antigen binding characteristics are discovered, the encoding DNA can be obtained. Library screening and use can incorporate ribosome display or phage display.

DNA sequencing refers to the process of determining the nucleotide order of a given DNA molecule. Generally, the sequencing can be performed using automated Sanger sequencing (e.g., using AB 13730x1 genome analyzer), pyrosequencing on a solid support (e.g., using 454 sequencing, Roche), sequencing-by-synthesis with reversible terminations (e.g., using ILLUMINA® Genome Analyzer), sequencing-by-ligation (e.g., using ABI SOLiD®) or sequencing-by-synthesis with virtual terminators (e.g., using HELISCOPE®) other next generation sequencing techniques for use with the disclosed methods include, Massively parallel signature sequencing (MPSS), Polony sequencing, Ion Torrent semiconductor sequencing, DNA nanoball sequencing, Heliscope single molecule sequencing, Single molecule real time (SMRT) sequencing, and Nanopore DNA sequencing.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following examples are provided to illustrate certain features and/or embodiments of the disclosure. This example should not be construed to limit the invention to the particular features or embodiments described.

### EXAMPLES

### Example 1

This example discloses development of a novel platform, referred to as DddA-sequencing (3D-seq), which provides a facile and sensitive approach to map DNA-protein interactions (DPIs) based on double-stranded DNA cytosine deaminases.

### Introduction

Nucleic acid-targeting deaminases are a diverse group of proteins that have found a number of biotechnological applications due to their ability to introduce mutations in DNA or RNA. Fusion of the single-stranded DNA (ssDNA) cytosine deaminase APOBEC to catalytically inactive or nickase variants of Cas9 led to the development of the first precision base editor capable of introducing single nucleotide substitutions (C⋅G-to-T⋅A) in vivo. This breakthrough technology inspired the repurposing of several other ssDNA and RNA-targeting deaminases as base editing tools, including editors that catalyze A⋅T-to-G⋅C substitutions in ssDNA, and RNA transcript editors that induce C to U or A to I modifications. RNA-targeting deaminases have additionally been employed for the identification of RNA-protein complex sites.

The bacterial toxin-derived cytosine deaminase, DddA, is unique as the only deaminase known to act preferentially on dsDNA. In the present Example, the dsDNA-targeting capability of DddA was harnessed in the development of 3D-seq, a new technique for genome-wide DPI mapping.

### Results and Discussion

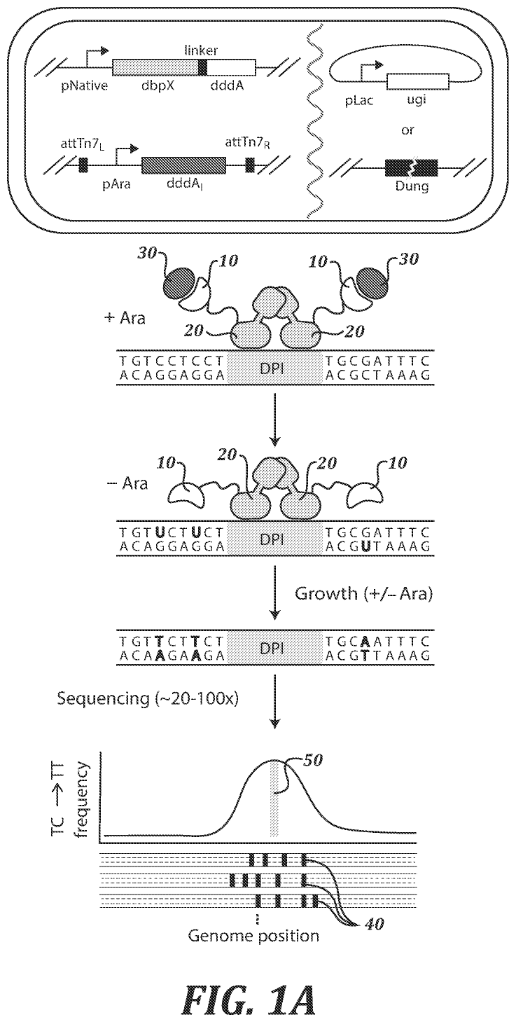

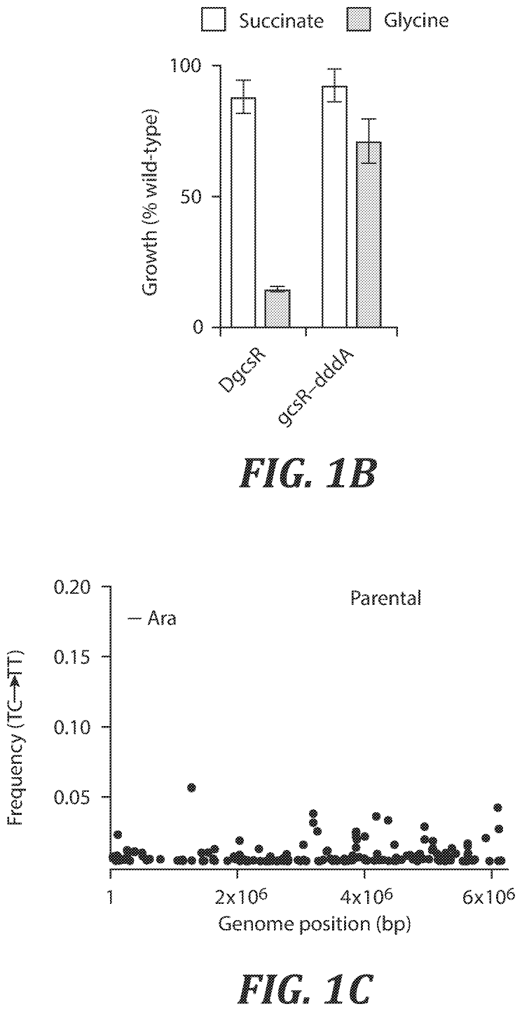

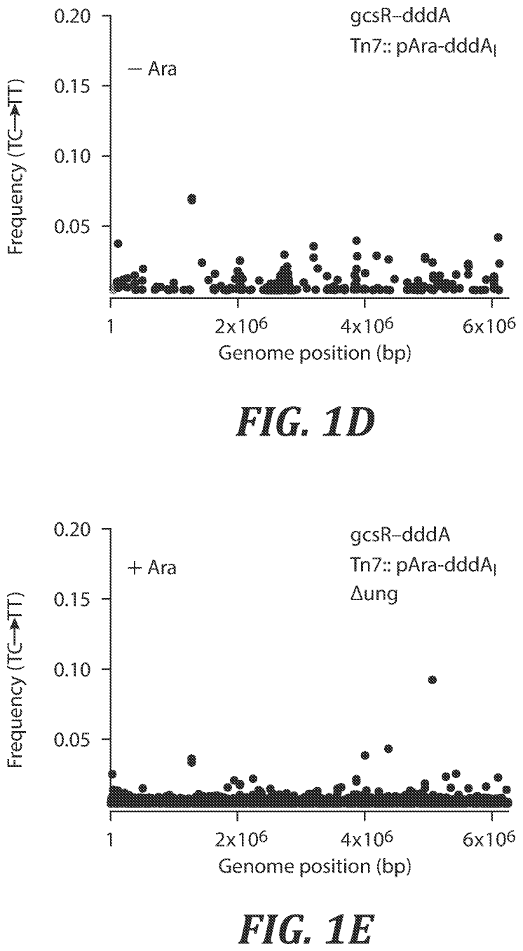

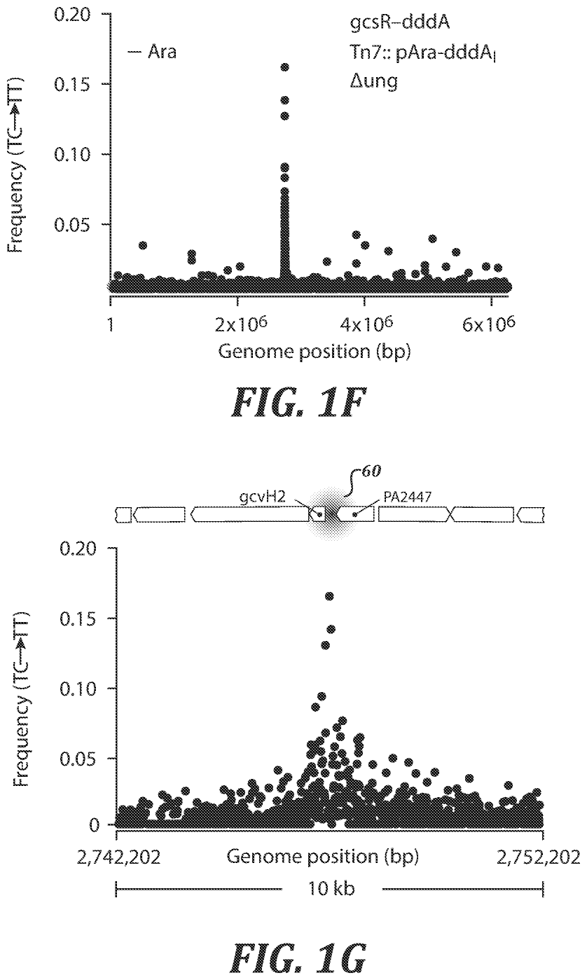

In DdCBEs, DddA activity is localized to particular sites on DNA by reconstitution of the enzymatic domain of the toxin (amino acids 1264-1427) from split forms fused to sequence-specific targeting proteins. An inverse approach was devised whereby fusion of the intact deaminase domain of DddA, referred to herein as DddA, to DBPs with unknown binding sites could be used to define sites of interaction (FIG. 1A). To test the feasibility of this approach, the candidate DNA binding protein GcsR of P. aeruginosa was selected. GcsR is a sigma 54-dependent transcription activator of an operon encoding the glycine cleavage system (gcvH2, gcvP2, and gcvT2) and auxiliary glycine and serine metabolic genes (glyA2 and sdaA) (Sarwar, Z. et al. GcsR, a TyrR-Like Enhancer-Binding Protein, Regulates Expression of the Glycine Cleavage System in Pseudomonas aeruginosa PAO1. mSphere 1, doi:10.1 128/mSphere.00020-16 (2016)). By analogy with closely related sigma 54-dependent regulators, also referred to as bacterial enhancer binding proteins (bEBPs), glycine binding to GcsR is thought to activate transcription of the operon by triggering conformational changes among subunits bound to three 18-bp tandem repeat binding sites in the gcvH2 promoter region. RNA-seq analyses of P. aeruginosa ΔgcsR suggest that the gcvH2 operon may encompass the only genes subject to direct regulation by GcsR (Sarwar, Z. et al. (2016), supra).

To capture physiologically relevant DNA binding, a GcsR-DddA translational fusion encoded at the native gcsR locus was generated. These efforts revealed that even in the context of fusion to transcription factors under native regulation, DddA exhibits sufficient toxicity to interfere with strain construction. To circumvent this, the gene encoding the DddA cognate immunity determinant, dddA_{I}, was inserted at the Tn7 attachment site under control of an arabinose inducible promoter (pAra). In this background, and with induction of immunity, gcsR was successfully replaced with an open reading frame encoding GcsR bearing an unstructured linker at its C-terminus fused to the deaminase domain of DddA (GcsR-DddA). Activation of the gcvH2 operon by GcsR is required for P. aeruginosa growth using glycine as a sole carbon source (Sarwar, Z. et al. (2016), supra). It was observed that, unlike a strain lacking GcsR, strains expressing GcsR-DddA effectively utilize glycine as a growth substrate, suggesting the fusion retains functionality (FIG. 1B).

It has been demonstrated that uracil DNA glycosylase (Ung) effectively inhibits uracil accumulation in cells exposed to DddA. Reasoning this DNA repair factor would limit the capacity to detect DddA activity, ung was deleted in the GcsR-DddA-expressing strain. Next, this strain was passaged in the presence and absence of arabinose and performed Illumina-based whole genome sequencing (WGS). Data from replicate experiments was minimally filtered to remove positions with low coverage or hypervariability (see methods) and the average frequency of C⋅G-to-T⋅A transition events within 5′-TC-3′ contexts were visualized across the P. aeruginosa genome (FIGS. 1C-1F). Other dinucleotide contexts were excluded based on the known strong preference of DddA for thymidine at the −1 position (Mok, B. Y. et al. A bacterial cytidine deaminase toxin enables CRISPR-free mitochondrial base editing. Nature 583, 631-637 (2020)). Remarkably, in samples propagated in the absence of arabinose, a single apparent peak of DddA activity was observed in this minimally filtered data, which was localized to the promoter region of gcvH2 (FIGS. 1F and 1G). This peak was not observed in samples containing arabinose, nor was it present in parallel studies using a strain containing Ung (FIGS. 1D and 1E).

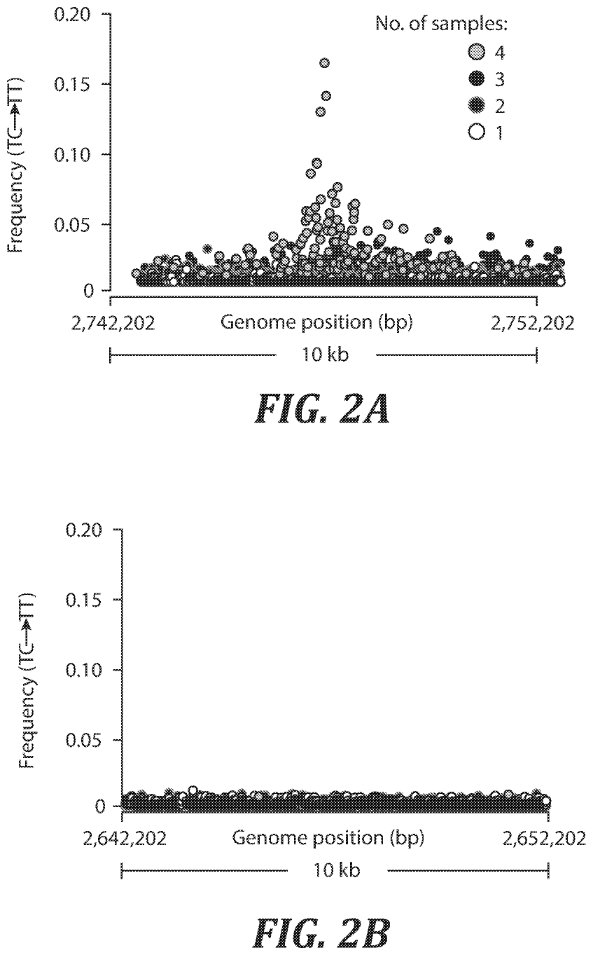

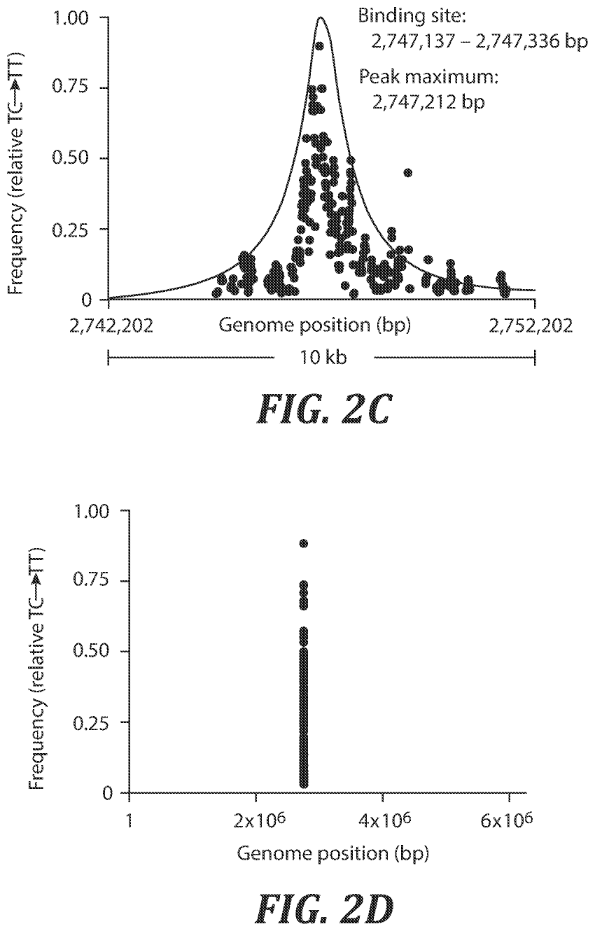

While a single peak of GcsR::DddA-dependent activity was readily apparent in the minimally processed data, it was reasoned that additional filtering to remove background signal would improve the sensitivity and accuracy of this technique. The filters employed are detailed in the methods and include i) accounting for sequencing errors by applying a minimum read count threshold for mutation events (˜1%), ii) eliminating positions lacking a neighboring transition event within the approximate length window likely to be accessible to a bound DBP-DddA fusion protein (100 bp), and iii) removing transitions representing SNPs present in the parent strain. Most significantly, given observation that modifications catalyzed by free DddA are randomly distributed across genomes, it was reasoned that substantial noise reduction could be achieved by removing transitions not reproduced in independent replicates. Visualization of four GcsR-DddA replicate datasets showed that transition events observed in at least three of the samples were highly enriched in the peak region associated with the gcvH2 promoter (FIGS. 2A and 2B), and therefore this criterion was added to the filtering workflow.

In parallel, a statistical analysis able to provide a quantitative means of distinguishing specific DPIs from background noise in 3D-seq data was developed. This approach employed a null hypothesis test and is described in detail in the methods. Briefly, a null hypothesis consisting of only background enzyme activity was compared to an alternative hypothesis in which a single putative peak was fit by maximum likelihood analysis. The null hypothesis was then either accepted or rejected at a confidence level of 95% using a Generalized Likelihood Ratio Test. If the null hypothesis was rejected, the model containing the peak replaced the null hypothesis and the test was repeated for another putative peak until no more peaks could be detected. P values for each detected peak are estimated and reported (TABLE 1). The application of these filtering criteria and statistical analyses to the GcsR 3D-seq data dramatically improved the apparent signal-to-noise and placed the major GcsR-DddA binding site centered within the 200 bp region containing the three known binding sites for GcsR (Sarwar, Z. et al. (2016), supra) (FIGS. 2C and 2D).

TABLE 1
Significant peaks detected in this study by 3D-seq.

| | | | | | |
|---|---|---|---|---|---|
| | | Peak | | | Closest |
| Peak | | maximum | Peak | Width | annotated |
| number¹ | p-value² | position | height³ | parameter⁴ | gene |

GcsR (Δung Tn7::pAra-dddA_{I} gcsR-dddA)

| | | | | | |
|---|---|---|---|---|---|
| 1 | <1.9E−999 | 2747212 | 0.0308 | 650 | gcvH2 |
| 2 | 4.4E−6 | 2754622 | 0.0032 | 507 | gcvH2 |

GacA (Δung ΔretS Tn7::pAra-dddA_{I} gacA-dddA)

| | | | | | |
|---|---|---|---|---|---|
| 1 | 2.0E−273 | 586705 | 0.0096 | 552 | rsmY |
| 2 | 4.5E−167 | 4057755 | 0.0090 | 449 | rsmZ |

FleQ (Δung Tn7::pAra-dddA_{I} fleQ-dddA)

| | | | | | |
|---|---|---|---|---|---|
| 1 | 1.9E−167 | 5192977 | 0.0182 | 828 | cdrA |
| 2 | 1.6E−157 | 2453477 | 0.0196 | 624 | pslA |
| 3 | 4.0E−25 | 3221641 | 0.0092 | 479 | PA2869 |
| 4 | 5.9E−25 | 1549819 | 0.0079 | 402 | bdlA |
| 5 | 1.6E−21 | 3434343 | 0.0067 | 704 | pelA |
| 6 | 2.2E−16 | 1592133 | 0.0066 | 563 | motD |
| 7 | 2.2E−16 | 1187368 | 0.0042 | 480 | fleQ |
| 8 | 5.7E−14 | 2129043 | 0.0054 | 529 | PA1945 |
| 9 | 2.4E−11 | 196854 | 0.0070 | 439 | siaA |
| 10 | 9.9E−11 | 1582818 | 0.0036 | 429 | flhF |
| 11 | 2.2E−09 | 2737599 | 0.0075 | 686 | PA2440 |
| 12 | 1.4E−04 | 1572170 | 0.0030 | 394 | fliL |
| 13 | 1.6E−02 | 3751994 | 0.0027 | 316 | PA3340 |
| 14 | 1.8E−02 | 1215200 | 0.0040 | 508 | yfiR |

GcsR (Tn7::pAra-dddA_{I} gcsR-dddA pPSV39::pLac-ugi)

| | | | | | |
|---|---|---|---|---|---|
| 1 | <1.9E−999 | 2747582 | 0.0830 | 511 | gcvH2 |
| 2 | 9.6E−113 | 6199779 | 0.0391 | 610 | PA5508 |
| 3 | 2.1E−83 | 2744696 | 0.0344 | 204 | gcvH2 |
| 4 | 1.5E−26 | 2141807 | 0.0153 | 1000 | PA1957 |
| 5 | 5.0E−22 | 6151145 | 0.0104 | 934 | PA5459 |
| 6 | 1.0E−21 | 4230516 | 0.0117 | 756 | PA3372 |
| 7 | 1.8E−21 | 2754908 | 0.0127 | 283 | PA2454 |
| 8 | 2.8E−17 | 1121658 | 0.0117 | 612 | PA1032 |
| 9 | 4.0E−15 | 2749090 | 0.0463 | 26 | PA2448 |
| 10 | 1.5E−12 | 6195909 | 0.0110 | 364 | PA5503 |
| 11 | 9.0E−12 | 2751477 | 0.0155 | 213 | PA2449 |
| 12 | 1.5E−09 | 2742380 | 0.0107 | 166 | PA2443 |
| 13 | 8.9E−08 | 2750504 | 0.0168 | 109 | gcsR |

¹Peaks are listed in order of increasing p-value.
²P-values calculated as described in the supplemental methods.
^{3,4}The peak profile function represents cell-mean allele frequency as a function of genomic position. The amplitude parameter I represents the height of the peak of the profile function. The width parameter L controls its width. See supplemental methods.

To benchmark the 3D-seq approach, a comparative study was performed using ChIP-seq—a current standard for assessing DPIs genome-wide in bacteria. In place of the dddA translational fusion at the 3′ end of gcsR, a sequence encoding the VSV-G epitope was inserted to facilitate the necessary immunoprecipitation step of ChIP-seq. Similar to 3D-seq, the most strongly supported candidate binding site for GcsR identified by ChIP-seq localized at the expected region upstream of gcvH2 (TABLE 2). In the course of this work it was noted that following strain construction, the 3D-seq workflow is considerably streamlined relative to that of ChIP-seq. The hands-on time to process a ChIP-seq sample to the point of sequencing library preparation is approximately one-week, whereas 3D-seq sample preparation constitutes only a genomic DNA preparation that occupies a portion of one day and requires little training.

TABLE 2
Significant peaks detected in this study by ChIP-seq.

| | | | | | |
|---|---|---|---|---|---|
| | | | | | Closest |
| Peak | Fold | Peak | Peak | Peak | annotated |
| number¹ | enrichment | maximum | start | end | gene |

GcsR (gcsR-VSV-G)

| | | | | | |
|---|---|---|---|---|---|
| 1 | 92 | 2747441 | 2746047 | 2748292 | gcvH2 |
| 2 | 13 | 5402788 | 5402462 | 5403174 | lipC |
| 3 | 9.1 | 945626 | 945487 | 945780 | PA0864 |
| 4 | 5.6 | 2516215 | 2516073 | 2516373 | PA2288 |
| 5 | 3.4 | 4373881 | 4373741 | 4374013 | PA3904 |
| 6 | 3.4 | 3070313 | 3070176 | 3070417 | PA2715 |
| 7 | 3.2 | 1205058 | 1204920 | 1205189 | PA1112a |

GacA (ΔretS gacA-VSV-G)

| | | | | | |
|---|---|---|---|---|---|
| 1 | 22E01 | 586983 | 586145 | 587484 | rsmY |
| 2 | 21E01 | 4057808 | 4057399 | 4058161 | rsmZ |
| 3 | 3.6 | 5214833 | 5214674 | 5215156 | PA4648 |

¹Peaks are listed in order of decreasing fold enrichment.

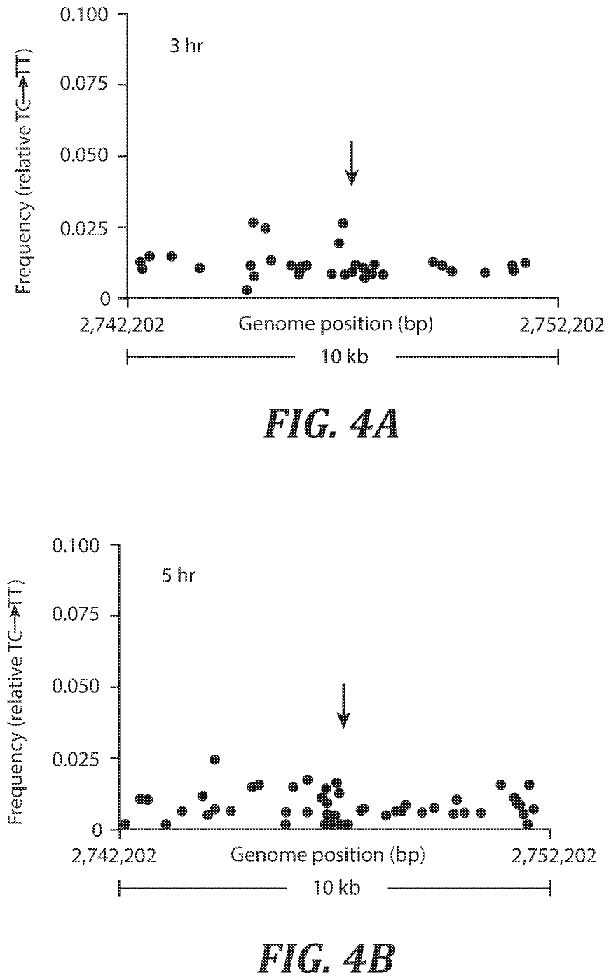

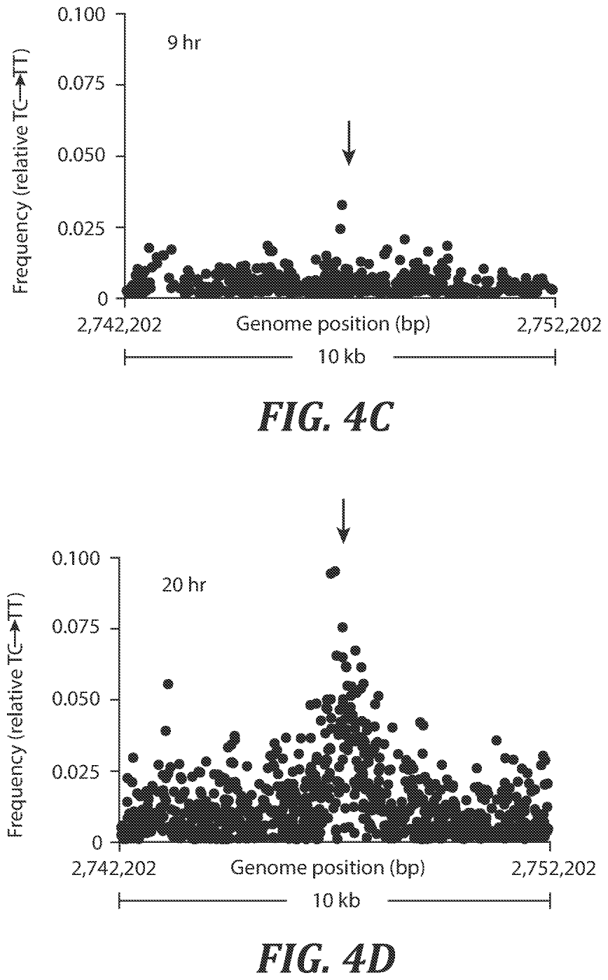

Given that the initial experiment for detecting GcsR-DddA-catalyzed mutagenesis involved growth for multiple passages, it was examined whether a peak of C⋅G-to-T⋅A transition frequency in the vicinity of the GcsR binding site could be detected after a shorter period of growth. In continuously growing cultures of P. aeruginosa Aung expressing GcsR-DddA in the absence of DddAᵣ induction a small peak was observed at 9 hrs of propagation and robust DddA-GcsR activity was detected at 20 hrs of growth (FIGS. 4A-4D). This latter incubation period was thus implemented for subsequent experiments.

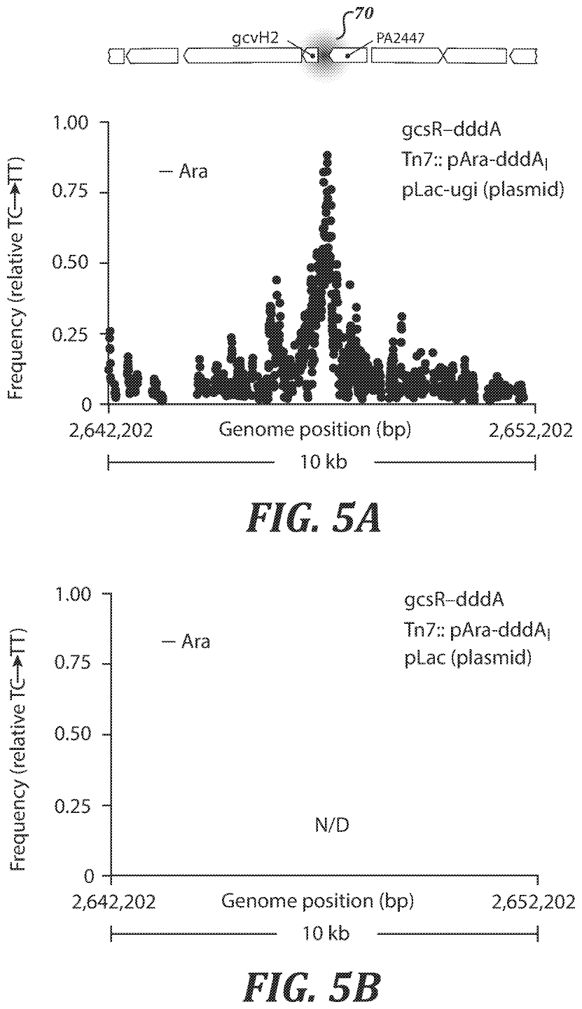

It was found that Ung inactivation is critical for the detection of GcsR-DNA interactions by 3D-seq (FIGS. 1E and 1F). As an alternative to a ung knockout, the question of whether expression of the Ung inhibitor protein, UGI (Mol, C. D. et al. Crystal structure of human uracil-DNA glycosylase in complex with a protein inhibitor: protein mimicry of DNA. Cell 82, 701-708), could achieve sufficient Ung inactivation to reveal GcsR DPIs was addressed. This approach is potentially advantageous for 3D-seq in organisms that are difficult to modify genetically. To determine whether expression of UGI could substitute for genetic inactivation of ung, P. aeruginosa expressing GcsR-DddA and DddA_{I} was supplied with a plasmid possessing Ugi under control of the lacUV5 promoter to allow orthogonal modulation of DddA_{I} (arabinose) and Ugi (IPTG). As when Ung was inactivated genetically, it was found that inhibition of Ung by UGI expression yielded a high significant peak of C⋅G-to-T⋅A transition events centered on the known GcsR binding site upstream of gcvH2 (FIGS. 5A and 5B, TABLE 1). This peak was not observed in the empty vector control strain.

To begin to probe the versatility of 3D-seq, the question of whether it could be successfully applied to the mapping of DPIs for a DBP that is structurally and functionally divergent from GcsR was investigated. For this analysis, the regulator GacA, which belongs to a large group of transcription factors known as response regulators, was selected. Canonically, phosphorylation of these proteins by cognate histidine kinases enhances their interaction with promoter elements, leading to modulation of transcription (Gao, R., Bouillet, S. & Stock, A. M. Structural Basis of Response Regulator Function. Annu. Rev. Microbiol. 73, 175-197 (2019)). In the case of GacA, phosphorylation by the sensor kinase GacS promotes binding of GacA to the promoter regions of two small RNA genes, rsmY and rsmZ (Lapouge, K., Schubert, M., Allain, F. H. & Haas, D. Gac/Rsm signal transduction pathway of gamma-proteobacteria. from RNA recognition to regulation of social behaviour. Mol. Microbiol. 67, 241-253 (2008)). GacS is itself regulated by a second sensor kinase, RetS, which strongly inhibits GacS phosphotransfer to GacA (Goodman, A. L. et al. Direct interaction between sensor kinase proteins mediates acute and chronic disease phenotypes in a bacterial pathogen. Genes Dev. 23, 249-259 (2009)). To further evaluate the capacity of 3D-seq to capture the effects of posttranslational regulation of a transcription factor, the studies were performed in both ΔgacS and ΔretS backgrounds of P. aeruginosa.

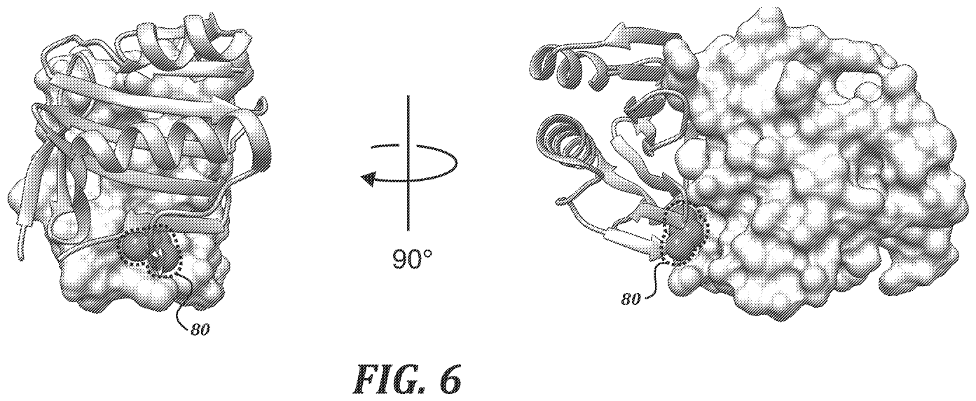

During preliminary testing of the 3D-seq protocol with GacA, it was found that repressing DddA_{I} production by removing arabinose did not lead to detectable DddA activity. It was reasoned that leaky expression of DddA_{I}, which is well documented to occur from pAra in P. aeruginosa, might be itself sufficient to effectively inhibit DddA in this instance. After exploring alternative promoters without success, a DddA₁ mutant was tested in which the interaction with DddA is weakened by a C-terminal FLAG epitope fusion (DddA_{I}-F, FIG. 6). At high arabinose levels, DddA_{I}-F provided sufficient protection against DddA to permit strain construction and under lower arabinose levels, DddA-dependent C⋅G-to-T⋅A transitions were observed.

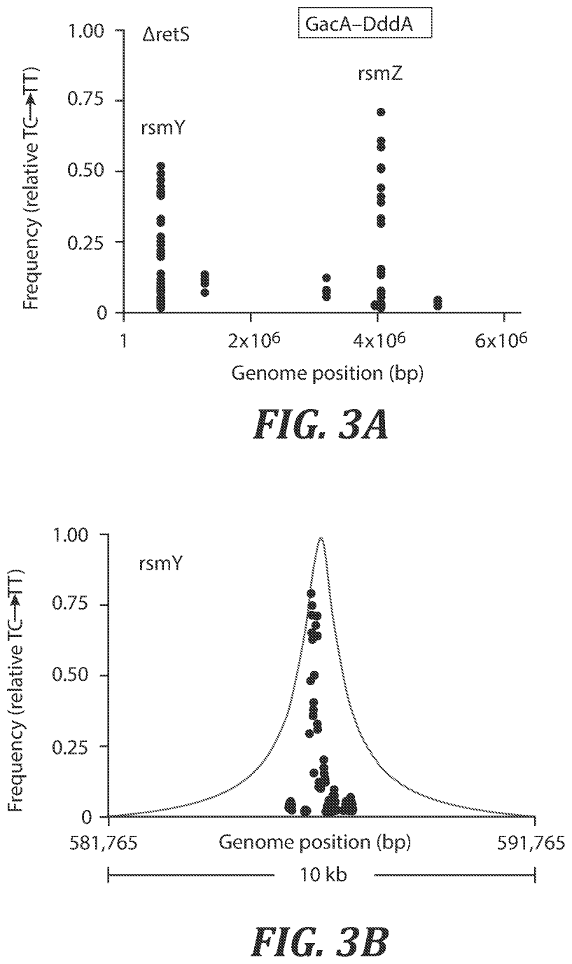

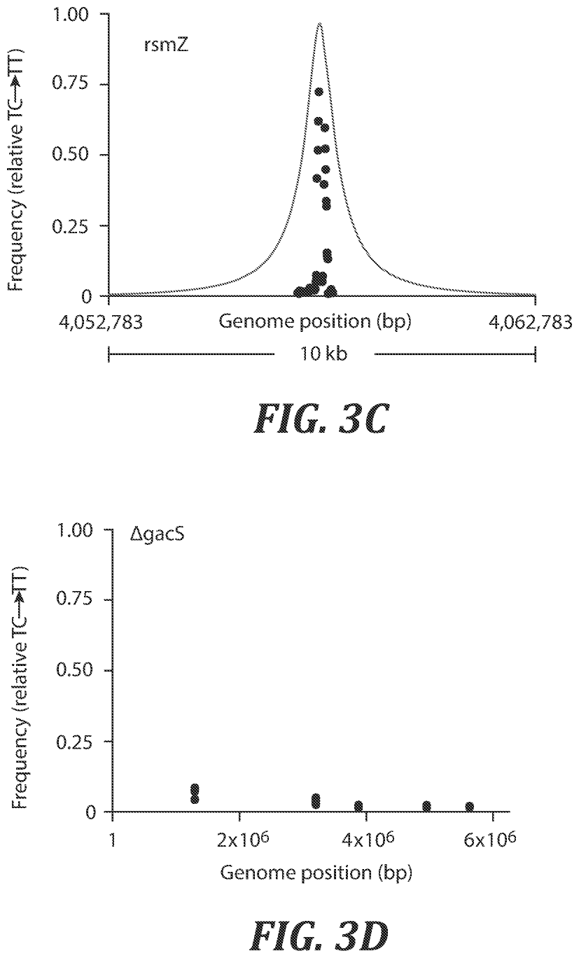

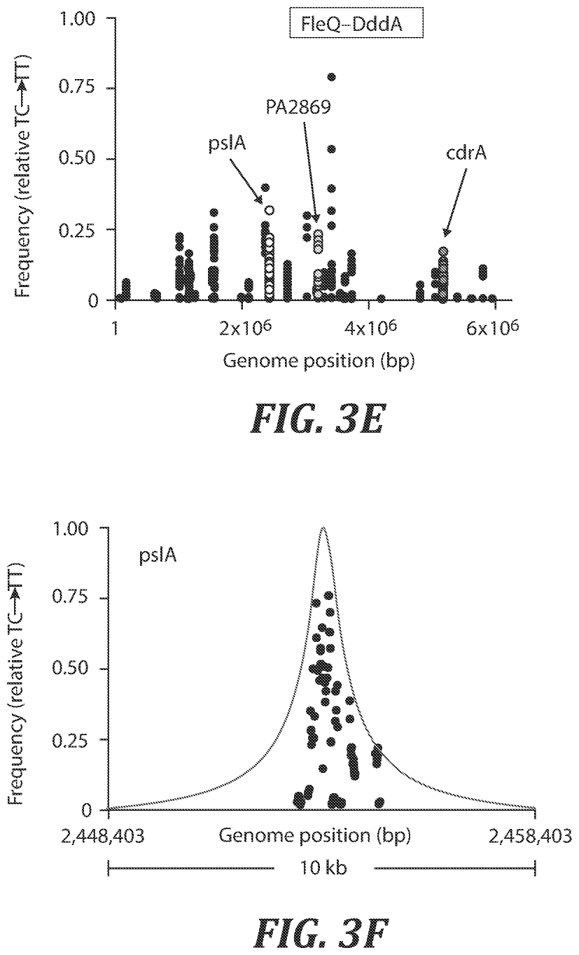

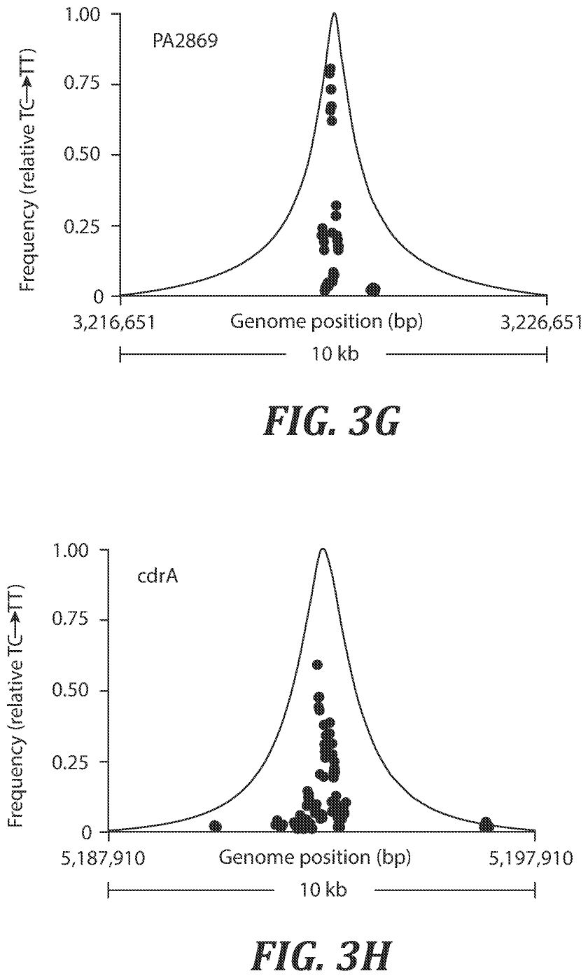

3D-seq revealed GacA binding sites upstream of rsmY and rsmZ in the ΔretS background of P. aeruginosa (FIGS. 3A-3C, TABLE 1). These peaks were the only significant GacA bindings sites detected and they were not found in the ΔgacS strain (FIG. 3D, TABLE 1). Huang et al. recently reported GacA binds 1125 sites across the P. aeruginosa genome, as measured by ChIP-seq (Huang, H. et al. An integrated genomic regulatory network of virulence-related transcriptional factors in Pseudomonas aeruginosa. Nat. Commun. 10, 2931 (2019)). Given the large discrepancy between this result and the present findings by 3D-seq, ChIP-seq analysis of GacA was performed in-house. Rather than over-express GacA, which was the strategy adopted by Huang et al., an epitope-tagged allele of the regulator was introduced at its native locus in the ΔretS background of P. aeruginosa. Consistent with the 3D-seq results and an earlier ChIP-ChIP study (Brencic, A. et al. The GacS/GacA signal transduction system of Pseudomonas aeruginosa acts exclusively through its control over the transcription of the RsmY and RsmZ regulatory small RNAs. Mol. Microbiol. 73, 434-445 (2009)), this approach identified regions upstream of rsmY and rsmZ, enriched 215- and 212-fold, respectively, as the two major bindings sites of GacA (TABLE 2). A third site located in the promoter region of PA4648 was the only additional site that surpassed the three-fold enrichment significance cut-off. These results added to confidence in 3D-seq-based DPI site identification and they showed that the methodology can be applied to regulators of different binding modalities and with multiple interaction sites. Finally, they show that 3D-seq can potentially be used to assess DPI dynamics under different regulatory states.

Although they represent different transcription factor families, these findings show that GcsR and GacA both interact with a limited number of sites on the P. aeruginosa chromosome. To gauge the performance of 3D-seq when applied to a DBP with many predicted sites of interaction, the regulator FleQ was selected. This protein is an unusual member of the bEBP family, as it can act as both an activator and repressor, it regulates transcription from both σ⁴ and σ⁷⁰-dependent promoters, and its regulatory functions appear to be modulated by interaction with an additional protein that does not bind DNA directly, FleN (Baraquet, C., et al. The FleQ protein from Pseudomonas aeruginosa functions as both a repressor and an activator to control gene expression from the pel operon promoter in response to c-di-GMP. Nucleic Acids Res. 40, 7207-7218 (2012); Dasgupta, N. et al. A four-tiered transcriptional regulatory circuit controls flagellar biogenesis in Pseudomonas aeruginosa. Mol. Microbiol. 50, 809-824 (2003); Jyot, J., et al. FleQ, the major flagellar gene regulator in Pseudomonas aeruginosa, binds to enhancer sites located either upstream or atypically downstream of the RpoN binding site. J. Bacteriol. 184, 5251-5260 (2002); and Hickman, J. W. & Harwood, C. S. Identification of FleQ from Pseudomonas aeruginosa as a c-di-GMP-responsive transcription factor. Mol. Microbiol. 69, 376-38 (2008)). In its capacity as σ⁵⁴-dependent transcription activator, studies have shown FleQ binds the promoters of several flagellar gene operons; as a σ⁷⁰-dependent regulator, it interacts with binding sites adjacent to or overlapping with transcription start sites for several genes involved in exopolysaccharide biosynthesis and can serve as both a repressor and activator depending on availability of the second messenger molecule cyclic-di-GMP (Baraquet, C., et al. (2012), supra; Jyot, J., et al. (2002), supra; and Baraquet, C. & Harwood, C. S. FleQ DNA Binding Consensus Sequence Revealed by Studies of FleQ-Dependent Regulation of Biofilm Gene Expression in Pseudomonas aeruginosa. J. Bacteriol. 198, 178-186 (2016)). To date, there are no published studies describing the full complement of genes directly regulated by FleQ in P. aeruginosa. FleQ was included in the study referenced above that utilized over-expressed transcription factors, but a list of FleQ sites was not provided, and the present GacA ChIP-seq and 3D-seq results suggest the general workflow adopted by the authors is problematic (Huang, H. et al. (2019), supra).

3D-seq analysis employing FleQ-DddA expressed from its native promoter identified 14 peaks with a significantly elevated frequency of C⋅G-to-T⋅A transition events (FIGS. 3E-3H, TABLE 1). Many of these peaks were localized to previously identified FleQ binding sites. Consistent with expectations for P. aeruginosa growing exponentially in liquid media, these included sites upstream of both exopolysaccaride biosynthesis and cell autoaggregation genes known to be repressed by FleQ (e.g. pelA, pslA, siaA) and flagellar motility genes known to be activated by the protein (e.g. flhF, fliL, motD) (Baraquet, C., et al. (2012), supra; Jyot, J., et al. (2002), supra; and Baraquet, C. & Harwood (2016), supra). Interestingly, significant peaks were also identified upstream of several uncharacterized genes, including a homolog of the motility genefimV (PA3340), a gene encoded upstream of a c-di-GMP biosynthetic enzyme (PA2869), and a gene with no predicted links to other FleQ-regulated functions (PA3440) (TABLE 1). These results illustrate the capacity of 3D-seq to sensitively and specifically identify DPIs for proteins that bind at many sites across the genome.

3D-seq represents the first known method for high-resolution genome-wide recording of DPIs in living cells. In addition to this unique capability of 3D-seq, the method was found to offer several advantages over commonly employed technologies for DPI mapping. Key among these is its ease in implementation. Once the appropriate genetic elements are in place, which can in principle be reduced to transformation by a single plasmid, 3D-seq involves simply growing a small volume of the strain under examination followed by genomic DNA preparation and standard WGS. In contrast, ChIP-seq requires a number of specialized reagents, including highly purified antibodies targeting the DBP of interest or an associated epitope tag, and the subsequent technically demanding immunoprecipitation procedure requires several days to complete. Another distinct advantage of 3D-seq is the minimal starting material required. Owing to handling challenges and sample loss occurring at each step of the ChIP-seq protocol, these experiments must generally be initiated with ˜40-80 mL of bacterial culture. The lower limit on material for a 3D-seq study is defined only by the terminal DNA sequencing technology being utilized. Indeed, it is believed that in many circumstances, the genome of a single cell would be adequate for revealing DPIs by 3D-seq.

As performed in this study, 3D-seq exploits the small size of bacterial genomes to cost-effectively obtain high coverage (>100-fold) that can be translated into semi-quantitative measures of DBP occupancy. It is also anticipated that 3D-seq will find application in organisms, e.g., eukaryotes, with large genomes. If experiments are conducted in a manner that permits mutations introduced by the DBP-DddA fusion of interest to approach 100% frequency in the population, far less sequencing depth is required. In another variation, candidate sites could be amplified by PCR and amplicon sequencing would be used to reveal lower frequency modifications.

Despite the strong performance of this initial demonstration of 3D-seq, there is ample opportunity for optimizing the technology. The straightforward genetic manipulation of P. aeruginosa allowed generation of chromosomally-encoded DBP-DddA fusions and DddA_{I} expression constructs. These sequences, along with that necessary for Ugi expression, could readily be incorporated into a single plasmid, thus eliminating the need for chromosomal manipulations.

The resolution of this implementation of 3D-seq was limited by the frequency of cytosines found in the sequence context preferred by DddA, 5′-TC-3′. In P. aeruginosa, this dinucleotide motif occurs on average every 12 bp, allowing sufficient resolution to accurately identify DPI sites. Although the average frequency of 5′-TC-3′ is expected to remain relatively consistent across organisms with varying GC content, within particular genomic regions, the frequency of 5′-TC-3′ could diminish substantially and limit resolution. DddA derivatives or novel dsDNA-targeting deaminases with alternative or relaxed sequence specificity (see e.g., de Moraes, M. H. et al. An interbacterial DNA deaminase toxin directly mutagenizes surviving target populations. Elife 10 (2021)) hold great promise as a solution to this limitation of 3D-seq.

While the utility of 3D-seq has been demonstrated for the population-level mapping of DPIs involving bacterial transcription factors during in vitro growth, its unique features will catalyze additional applications of the technology going forward. One such feature is the ability to modulate DddA activity through DddA_{I} expression, which enables 3D-seq to capture a snapshot of the DNA-protein landscape during a fixed period of time. This can be particularly advantageous for identifying DPIs during growth under physiological conditions inaccessible to other mapping methods, such as during colonization of a host. The capacity to inducibly inhibit DddA also raises the intriguing possibility of employing 3D-seq to map DPIs within single cells. In this embodiment of the technique, a bacterial population can be grown under a condition of interest in the absence of DddA_{I} expression, and subsequently individual clones would be isolated (e.g., as colonies) from media containing the inducer for DddA_{I}. Sequencing of these clones, which contain a mutational record of the activity and location of the DBP of interest, will provide heretofore unobtainable genome-wide insights into cell-cell heterogeneity in DPIs. In summary, the simplicity of 3D-seq will greatly improve the accessibility of genome-wide DPI mapping studies and its unique attributes will help usher in a new era of DPI measurements in physiological contexts.

### Methods

Bacterial Strains, Plasmids, and Growth Conditions

Detailed lists of all strains and plasmids used in this study can be found in TABLES 3 and 4. All P. aeruginosa strains were derived from the sequenced strain PAO1 (Stover, C. K. et al. Complete genome sequence of Pseudomonas aeruginosa PA01, an opportunistic pathogen. Nature 406, 959-964 (2000)) and were grown on Luria-Bertaini (LB) medium at 37° C. supplemented as appropriate with 30 pg ml⁻¹ gentamicin, 25 μg ml⁻¹irgasan, 5% (w v) sucrose, 1.0 mM IPTG (isopropyl β-D-1-thiogalactopyranoside), and arabinose at varying concentrations. Escherichia coli was grown in LB medium supplemented as appropriate with 15 μg ml⁻¹ gentamicin, 50 μg ml⁻¹ trimethoprim, and 1% rhamnose. E. coli strains DH5α was used for plasmid maintenance and SM10 (Novagen, Hornsby Westfield, Australia) HB101 (pRK2103) and S17-1 were used for conjugative transfer.

TABLE 3
Bacterial strains used

| Strain | Source |
|---|---|
| P. aeruginosa strains | |
| PAO1 | Stover, C. K. et al. Nature 406, |
| | 959-964 (2000) |
| Δung | de Moraes, M. H. et al. Elife 10 |
| | (2021) |
| attTn7::araC-P_{BAD}-dddA_{I} | This study |
| Δung attTn7::araC-P_{BAD}-dddA_{I} | This study |
| attTn7::araC-P_{BAD}-dddA_{I} gcsR-dddA | This study |
| Δung attTn7::araC-P_{BAD}-dddA_{I} gcsR-dddA | This study |
| attTn7::araC-P_{BAD}-dddA_{I} gcsR-dddA pPSV39(empty) | This study |
| attTn7::araC-P_{BAD}-dddA_{I} gcsR-dddA pPSV39-ugi | This study |
| attTn7::araC-P_{BAD}-dddA_{I}-FLAG | This study |
| Δung attTn7::araC-P_{BAD}-dddA_{I}-FLAG | This study |
| Δung attTn7::araC-P_{BAD}-dddA_{I}-FLAG gacA-dddA | This study |
| Δung attTn7::araC-P_{BAD}-dddA_{I}-FLAG gacA-dddA ΔretS | This study |
| Δung attTn7::araC-P_{BAD}-dddA_{I}-FLAG gacA-dddA ΔgacS | This study |
| Δung attTn7::araC-P_{BAD}-dddA_{I}-FLAG fleQ-dddA | This study |
| ΔgcsR | This study |
| GcsR-V | This study |
| ΔretS | This study |
| ΔretS GacA-V | This study |
| E. coli strains | |
| DH5α | |
| SM10 | Novagen |
| | Boyer, H. W. & Roulland-Dussoix, |
| HB101 | D. J. Mol. Biol. 41, 459-472 (1969) |
| S17-1 | Simon, R., et al. Nat. Biotechnol. 1, |
| | 784-791 (1983) |

TABLE 4
Plasmids used

| Plasmid | Reference |
|---|---|
| pEXG2 | Rietsch, A., et al. Proc. Natl. Acad. Sci. U.S.A. |
| | 102, 8006-8011 (2005) |
| pSCrhaB2 | Cardona, S. T. & Valvano, M. A. Plasmid 54, |
| | 219-228 (2005) |
| pPSV39 | Silverman, J. M. et al. Mol. Cell 51, 584-593 |
| | (2013) |
| pUC18T-miniTn7T-Gm-pBAD-araE | Kulasekara, B. R. et al. Elife 2, e01402 (2013) |
| pRK2103 | Figurski, D. H. & Helinski, D. R. Proc. Natl. |
| | Acad. Sci. U.S.A. 76, 1648-1652 (1979) |
| pTNS3 | Choi, K. H. et al. Appl. Environ. Microbiol. |
| | 74, 1064-1075 (2008) |
| pFLP2 | Hoang, T. T., et al. Gene 212, 77-86 (1998) |
| pEXG2-Δung | de Moraes, M. H. et al. Elife 10 (2021) |
| pEXG2-ΔretS (pEXG2_ΔPA4856) | Mougous, J. D. et al. Science 312, 1526-1530 |
| | (2006) |
| pEXG2-ΔgacS (pEXG2_ΔPA0928) | LeRoux, M. et al. Elife 4 (2015) |
| pSCrhaB2-dddA_{I} | This study |
| pEXG2-gcsR-dddA | This study |
| pEXG2-gacA-dddA | This study |
| pEXG2-fleQ-dddA | This study |
| pEXG2-ΔgcsR | This study |
| pEXG2-GsCR-V | This study |
| pEXG2-GacA-V | This study |
| pPSV39-ugi | This study |
| pUC18T-miniTn7T-Gm-pBAD-araE-dddA_{I} | This study |
| pUC18T-miniTn7T-Gm-pBAD-araE-dddA_{I}-FLAG | This study |

Plasmid Construction

Details of plasmid construction and primer sequences are provided in TABLES 5 and 6. Plasmid pEXG2 was used to make the in-frame deletion constructs pEXG2-ΔgcsR as well as the VSV-G insertion constructs pEXG2-GcsR-V and pEXG2-GacA-V and the DddA fusion constructs pEXG2-gcsR:: dddA, pEXG2-gacA-dddA and pEXG2-fleQ-dddA (Rietsch, A., et al. ExsE, a secreted regulator of type III secretion genes in Pseudomonas aeruginosa. Proc. Natl. Acad. Sci. U.S.A 102, 8006-8011 (2005)). Plasmid pEXG2-ΔgcsR, was constructed by amplification of ˜400 bp regions of genomic DNA flanking gcsR, with primers containing restriction sites, followed by digestion and ligation into pEXG2 that had been digested with the appropriate restriction enzymes. C-terminal VSV-G insertion constructs for GcsR-V and GacA-V were made by amplifying ˜400 bp regions flanking each insertion site using primers that contained an in-frame sequence encoding the VSV-G epitope tag. Constructs for generating DddA fusions encoded a protein in which DddA was fused to the C-terminus via a 32aa linker, (SGGSSGGSSGSETPGTSESATPESSGGSSGGS (SEQ ID NO:6). To generate these constructs, primers with 3′ overlapping regions were used to amplify both the linker and dddA, as well as 500 bp regions flanking the C-terminus of each gene. Gibson assembly (Gibson, D. G. et al. Enzymatic assembly of DNA molecules up to several hundred kilobases. Nat Methods 6, 343-345 (2009)) was then used for the generation of the pEXG2 plasmids containing each construct, and assembly mixes were transformed into E. coli DH5α expressing DddA_{I} from pSCrhaB2-DddA_{I} to avoid DddA-mediated toxicity. Construction of pEXG2-derived plasmids for deletion of gacS, retS and ung was previously described (de Moraes, M. H. et al. An interbacterial DNA deaminase toxin directly mutagenizes surviving target populations. Elife 10 (2021); LeRoux, M. et al. Kin cell lysis is a danger signal that activates antibacterial pathways of Pseudomonas aeruginosa. Elife 4 (2015); Mougous, J. D. et al. A virulence locus of Pseudomonas aeruginosa encodes a protein secretion apparatus. Science 312, 1526-1530 (2006)). Site-specific chromosomal insertions of the immunity gene dddA_{I}(with or without a FLAG tag at encoded at the C-terminus) were generated using pUC18T-miniTn7T-Gm-pBAD-araE. The genes encoding DddA_{I} or DddA_{I}-FLAG were amplified and cloned into the KpnI/HindIII sites of this vector through Gibson assembly, to generate pUC18-miniTn7T-Gm-pBAD-araE-dddA_{I} and pUC18T-miniTn7T-Gm-pBAD-araE-dddA_{I}-FLAG.

TABLE 5
Plasmid construction. The following plasmids were generated by combining the
individual fragments listed using either Gibson cloning or overlap extension PCR.
Primer sequences are listed in Table 6.

| | | | |
|---|---|---|---|
| Fragment | Template | F primer | R primer |

pSCrhaB2-dddA_{I}

| | | | |
|---|---|---|---|
| pSCrhaB2/NdeI/XbaI | NA | | |
| dddA_{I} | dddA_{I} ¹³ | pSCRhaB::dddA_{I}-F | pSCRhaB::dddA_{I}-R |

pEXG2-gcsR-dddA

| | | | |
|---|---|---|---|
| pEXG2/HindIII/XhoI | NA | | |
| 5′ end of gcsR | PAO1 gDNA | gcsR-dddA 1 | gcsR-dddA 2 |
| linker + dddA | XTEN linker ⁵⁰ | gcsR-dddA 3 | gcsR-dddA 4 |
| gcsR 3′ flank | PAO1 gDNA | gcsR-dddA 5 | gcsR-dddA 6 |

pEXG2-gacA-dddA

| | | | |
|---|---|---|---|
| pEXG2/HindIII/XhoI | NA | | |
| 5′ end of gacA | PAO1 gDNA | gacA-dddA 1 | gacA-dddA 2 |
| linker + dddA | XTEN linker ⁵⁰ | gacA-dddA 3 | gacA-dddA 4 |
| gacA 3′ flank | PAO1 gDNA | gacA-dddA 5 | gacA-dddA 6 |

pEXG2-fleQ-dddA

| | | | |
|---|---|---|---|
| pEXG2/HindIII/XhoI | NA | | |
| 5′ end of fleQ | PAO1 gDNA | fleQ-dddA 1 | fleQ-dddA 2 |
| linker + dddA | XTEN linker ⁵⁰ | fleQ-dddA 3 | fleQ-dddA 4 |
| fleQ 3′ flank | PAO1 gDNA | fleQ-dddA 5 | fleQ-dddA 6 |

pUC18T-miniTn7T-Gm-p_{BAD}-araE-dddAI

| | | | |
|---|---|---|---|
| pUC18T-miniTn7T-Gm- | NA | | |
| pBAD-araE/KpnI/HindIII | | | |
| dddA_{I} | dddA_{I} ¹³ | pUC18-dddA_{I}-F | pUC18-dddA_{I}-R |

pUC18T-miniTn7T-Gm-p_{BAD}-araE-dddAI-FLAG

| | | | |
|---|---|---|---|
| pUC18T-miniTn7T-Gm- | NA | | |
| pBAD-araE/KpnI/HindIII | | | |
| dddA_{I}-FLAG | dddA_{I} ¹³ | PUC18-dddA_{I}-F | pUC18-dddA_{I}-FLAG-R |

pPSV39-ugi

| | | | |
|---|---|---|---|
| pPSV39/SacI/XbaI | NA | | |
| ugi | ugi gBlock codon | pPSV39-UGI-F | pPSV39-UGI-R |
| | optimized | | |

pEXG2-ΔgcsR

| | | | |
|---|---|---|---|
| pEXG2/HindIII/BamHI | NA | | |
| gcsR 5′ flank | PAO1 gDNA | pEX.del.gcsR 1 | pEX.del.gcsR 2 |
| gcsR 3′ flank | PAO1 gDNA | pEX.del.gcsR 3 | pEX.del.gcsR 4 |

pEXG2-GcsR-V

| | | | |
|---|---|---|---|
| pEXG2/HindIII/BamHI | NA | | |
| gacA 5′ flank | PAO1 gDNA | pEX-GcsR-V 1 | pEX-GcsR-V 2 |
| gacA 3′ flank | PAO1 gDNA | pEX-GcsR-V 3 | pEX-GcsR-V 4 |

pEXG2-GacA-V

| | | | |
|---|---|---|---|
| pEXG2/HindIII/BamHI | NA | | |
| fleQ 5′ flank | PAO1 gDNA | pEX-GacA-V 1 | pEX-GacA-V 2 |
| fleQ 3′ flank | PAO1 gDNA | pEX-GacA-V 3 | pEX-GacA-V 4 |

TABLE 6
Primer sequences

| Primer | Sequence | SEQ ID NO: |
|---|---|---|
| pSCRhaB::dddAI-F | TGAAATTCAGCAGGATCACATATGTACGCAGACGATTTCGAC | 8 |
| pSCRhaB::dddAI-R | TCATTTCAATATCTGTATATCTAGATTACAACTCGCTCCATGTC | 9 |
| gcsR-dddA 1 | GGAAGCATAAATGTAAAGCAAGCTTGCAACCTGGAGAAGATGGTCGCCG | 10 |
| gcsR-dddA 2 | TACCTCCAGAGGCGCGCGGACCGATGCC | 11 |
| gcsR-dddA 3 | TCCGCGCGCCTCTGGAGGTAGCTCCGGC | 12 |
| gcsR-dddA 4 | GCCCGCTTCAACAACCTCCTTTCGTGGG | 13 |
| gcsR-dddA 5 | AGGAGGTTGTTGAAGCGGGCTCAGCCCT | 14 |
| gcsR-dddA 6 | TTAAGGTACCGAATTCGAGCTCGAGCAATCCCAAGGAGTTCGAGCG | 15 |
| gacA-dddA 1 | GGAAGCATAAATGTAAAGCAAGCTTCGGATGTCGTCCTGATGGAC | 16 |
| gacA-dddA 2 | TACCTCCAGAGCTGGCGGCATCGACCAT | 17 |
| gacA-dddA 3 | TGCCGCCAGCTCTGGAGGTAGCTCCGGC | 18 |
| gacA-dddA 4 | CGCTCATCTAACAACCTCCTTTCGTGGG | 19 |
| gacA-dddA 5 | AGGAGGTTGTTAGATGAGCGCCGTTTTCGACGC | 20 |
| gacA-dddA 6 | TTAAGGTACCGAATTCGAGCTCGAGGGCCGCGTACGGTTGCGG | 21 |
| fleQ-dddA 1 | GGAAGCATAAATGTAAAGCAAGCTTTCGCCCTGCTGCTCAACG | 22 |
| fleQ-dddA 2 | TACCTCCAGAATCATCCGACAGGTCGTCG | 23 |
| fleQ-dddA 3 | GTCGGATGATTCTGGAGGTAGCTCCGGC | 24 |
| fleQ-dddA 4 | CGACCTGTCAACAACCTCCTTTCGTGGG | 25 |
| fleQ-dddA 5 | AGGAGGTTGTTGACAGGTCGTTTCGCAACGCTTTG | 26 |
| fleQ-dddA 6 | TTAAGGTACCGAATTCGAGCTCGAGCGCGCGGAGCGAAGCAGC | 27 |
| pPSV39-UGI-F | GATAACAATTTCAGAATTCGAGCTCACGGGAGGAAAGATGACGAATCTC | 28 |
| | AGCGACAT | |
| pPSV39-UGI-R | TCATTTCAATATCTGTATATCTAGATTAGAGCATCTTGATTTTGTTCTC | 29 |
| | GC | |
| pUC18-dddAI-F | GGGCTAGCGAATTCGAGCTCGGTACCACGGGAGGAAAGATGTAC | 30 |
| pUC18-dddAI-R | CTCATCCGCCAAAACAGCCAAGCTTTCACAACTCGCTCCATGTCCTTCT | 31 |
| pUC18-dddAI- | CTCATCCGCCAAAACAGCCAAGCTTTCATTTGTCGTCGTCGTCTTTGTA | 32 |
| FLAG-R | GTCCAACTCGCTCCATGTCAG | |
| pEX.del.gcsR 1 | CATAAATGTAAAGCAAGCTTGGTACCGAGGCGGACT | 33 |
| pEX.del.gcsR 2 | AGCCCGCTTCAGGCGCGCGGGATGCGCATGCGGGA | 34 |
| pEX.del.gcsR 3 | CAGGTTCCCGCATGCGCATCCCGCGCGCCTGAAGC | 35 |
| pEX.del.gcsR 4 | TCGAGCTCGAGCCCGGGGATCCTTCGATTACCCACCTGC | 36 |
| pEX-GcsR-V 1 | CATAAATGTAAAGCAAGCTTACCTGTTCTACCGCCTCA | 37 |
| pEX-GcsR-V 2 | CTTGCCGAGGCGGTTCATTTCGATGTCGGTGTAAGCGGCCGCGGCGCGC | 38 |
| | GGACCGATGC | |
| pEX-GcsR-V 3 | GCGGCCGCTTACACCGACATCGAAATGAACCGCCTCGGCAAGTGAAGCG | 39 |
| | GGCTCAGCCC | |
| pEX-GcsR-V 4 | TCGAGCTCGAGCCCGGGGATCCGAGTTCGAGCGCTTCAG | 40 |
| pEX-GacA-V 1 | CATAAATGTAAAGCAAGCTTGAACTGAAGCCGGATGTC | 41 |
| pEX-GacA-V 2 | CTTGCCGAGGCGGTTCATTTCGATGTCGGTGTAAGCGGCCGCGCTGGCG | 42 |
| | GCATCGACCA | |
| pEX-GacA-V 3 | GCGGCCGCTTACACCGACATCGAAATGAACCGCCTCGGCAAGTAGATGA | 43 |
| | GCGCCGTTTTC | |
| pEX-GacA-V 4 | TCGAGCTCGAGCCCGGGGATCCGCGCTCGGATAGGGACC | 44 |

Strain Construction

P. aeruginosa strains containing in-frame deletions of gcsR, ung, retS or gacS were constructed by allelic replacement using the appropriate pEXG2-derived deletion construct and were verified by PCR and site specific or genomic sequencing as described previously (Rietsch, A., et al. ExsE, a secreted regulator of type III secretion genes in Pseudomonas aeruginosa. Proc. Natl. Acad. Sci. U.S.A 102, 8006-8011 (2005)). P. aeruginosa cells synthesizing GcsR with a C-terminal VSV-G epitope tag from the native chromosomal location were made by allelic replacement using vector pEXG2-GcsR-V. P. aeruginosa ΔretS mutant cells synthesizing GacA with a C-terminal VSV-G epitope tag from the native chromosomal location (P. aeruginosa ΔretS GacA-V) were made by allelic replacement using vector pEXG2-GacA-V. The P. aeruginosa ΔgcsR, GcsR-V, and ΔretS GacA-V strains were verified by PCR and production of the GcsR-V and GacA-V fusion proteins was verified by Western blotting using an antibody against the VSV-G epitope tag. P. aeruginosa strains producing DddA fusion proteins were generated by first engineering the parent strain to express DddA_{I} or DddA_{I}-FLAG from the chromosome under arabinose-inducible control by introduction of pUC18T-miniTn7T-Gm-pBAD-araE-dddA_{I} or pUC18T-miniTn7T-Gm-pBAD-araE-dddA_{I}-FLAG and helper plasmids pTNS3 and pRK2013 via tetraparental mating (Kulasekara, B. R. et al. c-di-GMP heterogeneity is generated by the chemotaxis machinery to regulate flagellar motility. Elife 2, e01402 (2013)). After chromosomal integration the GmR marker was removed from these cassettes by Flp/FRT recombination using plasmid pFLP2, which was then cured by sucrose counterselection (Hoang, T. T., et al. Integration-proficient plasmids for Pseudomonas aeruginosa: site-specific integration and use for engineering of reporter and expression strains. Plasmid 43, 59-72 (2000)). P. aeruginosa strains synthesizing GcsR-DddA, GacA-DddA or FleQ-DddA from the native chromosomal loci of each regulator were then generated by two-step allelic exchange using the relevant pEXG2 construct. Rhamnose (0.1%, for E. coli) or arabinose (0.1%, for P. aeruginosa) were maintained during the DddA-fusion expressing strain construction process to minimize DddA toxicity and off-target activity. Fusion-expressing strains were verified by PCR and by assembly of complete genome sequences obtained during 3D-seq analyses.

Assessing the Functionality of the GcsR-DddA Fusion Protein

To determine the functionality of the GcsR-DddA fusion protein cells were grown in biological triplicate in No Carbon E (NCE) minimal media (Davis, R. W., et al. Advanced Bacterial Genetics: A Manual for Genetic Engineering. (Cold Spring Harbor Laboratory, 1980)) containing arabinose (1%) and glycine (20 mM), or arabinose (1%) and succinate (20 mM), at 37° C. with aeration for 48 hours. Growth was determined by measuring the culture OD₆₀₀.

3D-Seq Sample Preparation and Sequencing

Culturing of DddA-Fusion Expressing Strains

To generate genomic DNA for 3D-Seq analysis, strains carrying specific DddA fusion constructs and attTn7::araC-P_{BAD}-dddI (GcsR) or attTn7::araC-P_{BAD}-dddI-FLAG (GacA, FleQ) were grown for varying amounts of time and with variable levels of arabinose to induce DddA_{I} or DddA_{I}-FLAG expression and/or IPTG to induce UGI production from pPSV39-UGI. In each case, the strains were initially streaked for single colonies on LB containing 0.1% or 1% arabinose, and single colonies were used to inoculate quadruplicate liquid cultures containing 0.1% or 1% arabinose. After ˜16 hrs growth, these cultures were then washed with LB and used to inoculate fresh cultures. For GcsR-DddA in Δung and ung+ backgrounds and for the Δung strain without a dddA fusion construct, washed cultures were inoculated into LB containing 0.1% (negative control) or no (experimental) arabinose at OD₆₀₀=0.02, then grown for 8 hrs before diluting back to OD₆₀₀=0.02. After an additional ˜16 hrs, cultures were again washed and diluted to OD₆₀₀=0.02, then grown a final 8 hrs before samples were collected for genomic DNA preparation. For gacA-dddA (with ΔretS or ΔgacS) and fleQ-dddA, washed cultures were inoculated into LB containing 0.0005% arabinose at OD₆₀₀=0.02, then grown for 6.5 hrs before samples were collected for genomic DNA preparation.

Genomic DNA Preparation and Sequencing

Genomic DNA was isolated from bacterial pellets using DNEasy Blood and Tissue Kit (Qiagen). Sequencing libraries for whole-genome sequencing were prepared from 200-300 ng of DNA using DNA Prep Kit (Illumina), with KAPA HiFi Uracil+ Kit (Roche) used in place of Enhanced PCR Mix for the amplification step. Libraries were sequenced in multiplex by paired-end 150-bp reads on NextSeq 550 and iSeq instruments (Illumina).

ChIP-Seq Sample Preparation and Library Construction

200 mL cultures of the P. aeruginosa GcsR-V, wild-type, ΔretS and ΔretS GacA-V strains were grown in biological triplicate to an OD₆₀₀ of 1.5 in LB at 37° C. with aeration. 80 mL of culture was crosslinked with formaldehyde (1%) for 30 minutes at room temperature with gentle agitation. Crosslinking was quenched by the addition of glycine (250 mM) and cells were incubated at room temperature for 15 minutes with gentle agitation. Cells were pelleted by centrifugation, washed three times with phosphate buffered saline, and stored at −80° C. prior to subsequent processing. Cell pellets were resuspended in 1 mL Buffer 1 (20 mM KHEPES, pH 7.9, 50 mM KCl, 0.5 mM dithiothreitol, 10% glycerol) plus protease inhibitor (complete-mini EDTA-free (Roche); 1 tablet per 10 mL), diluted to a total volume of 5.2 mL and divided equally among four 15 mL conical tubes (Corning). Cells were subsequently lysed and DNA sheared in a Bioruptor water bath sonicator (Diagenode) by exposure to two 8-minute cycles (30 seconds on, 30 seconds off) on the high setting. Cellular debris was removed by centrifugation at 4° C. for 20 minutes at 20,000×g. Cleared lysates were adjusted to match the composition of the immunoprecipitation (IP) buffer (10 mM Tris-HCl, pH 8.0, 150 mM NaCl, 0.1% NP-40 alternative (EMD-Millipore product 492018). The adjusted lysates were combined with anti-VSV-G agarose beads (Sigma) that had been washed once with IP buffer and reconstituted to a 50/50 bead/buffer slurry. For IP, 75 μL of the washed anti-VSV-G beads were added to each of the four aliquots for a given sample. IP was performed overnight at 4° C. with gentle agitation. Beads were then washed 5 times with 1 mL IP buffer and 2 times with 1X TE buffer (10 mM Tris-HCl, pH 7.4, 1 mM EDTA). Immune complexes were eluted from beads by adding 150 μL of TES buffer (50 mM Tris-HCl pH 8.0, 10 mM EDTA, 1% Sodium Dodecyl Sulfate (SDS)) and heating samples to 65° C. for 15 minutes. Beads were pelleted by centrifugation (5 minutes at 16,000×g) at room temperature and a second elution was performed with 100 μL of 1X TE+1% SDS. Supernatants from both elution steps were combined and incubated at 65° C. overnight to allow cross-link reversal. DNA was then purified with a PCR purification kit (QIAGEN), eluted in 55 μL of 0.1X Elution Buffer and quantified on an Agilent Bioanalyzer. ChIP-Seq libraries were prepared from 1-40 ng of DNA using the NEBNext Ultra II DNA Library Prep Kit for Illumina (NEB). Adaptors were diluted 10-fold prior to ligation. AMPure XP beads (Beckman Coulter) were used to purify libraries, which were subjected to 7 rounds of amplification without size selection. Libraries were sequenced by the Biopolymers Facility (Harvard Medical School) on an Illumina HiSeq2500 producing 75-bp paired-end reads (Gebhardt, M. J., et al. Widespread targeting of nascent transcripts by RsmA in Pseudomonas aeruginosa. Proc. Natl. Acad. Sci. U.S.A. 117, 10520-10529 (2020)).

ChIP-Seq Data Analysis

ChIP-Seq data were analyzed as described previously (Gebhardt, M. J., et al. (2020), supra). Paired-end reads corresponding to fragments of 200 bp or less were mapped to the PAO1 genome (NCBI RefSeq NC_002516) using bowtie2 version 2.3.4.3 (Langmead, B. & Salzberg, S. L. Fast gapped-read alignment with Bowtie 2. Nat Methods 9, 357-359 (2012)). Only read 1 from each pair of reads was extracted and regions of enrichment were identified using QuEST version 2.4 (Valouev, A. et al. Genome-wide analysis of transcription factor binding sites based on ChIP-Seq data. Nat Methods 5, 829-834 (2008)). Reads collected from the PAO1 replicates (i.e. IP from PAO1 cells that do not synthesize any VSV-G tagged protein) were merged and served as the mock control for the reads from each of the PAO1 GcsR-V replicates. Merged reads from the PAO1 ΔretS replicates served as the mock control for the reads from the PAO1 ΔretS GacA-V replicates. The mock control data were used to determine the background for each corresponding ChIP biological replicate. The following criteria were used to identify regions of enrichment (peaks): (i) they must be 3.5-fold enriched in reads compared to the background, (ii) they are not present in the mock control, (iii) they have a positive peak shift and strand correlation, and (iv) they have a q-value of less than 0.01. Peaks of enrichment for GcsR-V and GacA-V were defined as the maximal region identified in at least two biological replicates. Data were visualized using the Integrative Genomics Viewer (IGV) version 2.5.0 (Thorvaldsdottir, H., et al. Integrative Genomics Viewer (IGV): high-performance genomics data visualization and exploration. Brief Bioinform 14, 178-192 (2013)). Peak analyses used BEDtools version 2.27.1.

3D-Seq Data Analysis

Fastq reads were first pre-processed using the HTStream pipeline v. 1.3.0 (s4hts.github.io/HTStream/), where the chain of programs is hts_SuperDeduper->hts_SeqScreener->hts_AdapterTrimmer->hts_QWindowTrim->hts_LengthFilter->hts_Stats. In each case logging was enabled and default settings were used, with the following exceptions: 1) For hts_QWindowTrim a window size of 20 bp was used with a minimum quality score of 10. 2) For hts_LengthFilter the minimum length was set to half the mean read length. Reads were subsequently aligned to the PAO1-UW reference sequence (ncbi.nlm.nih.gov/nuccore/NC_002516.2) using Minimap2 v. 2.17-r974-dirty (lh3.github.io/minimap2/) and the alignments were saved into sorted BAM files with SAMTools v. 1.10 (www.htslib.org/). Alignment position counts were then enumerated using PySAM v. 0.16.0.1 (pysam.readthedocs.io/en/latest/) using these settings: read_callback=‘all’, quality_threshold=20. The reference genome was then surveyed using Biopython v. 1.78 (biopython.org) to determine the proportion of high-quality read-pairs covering each 5′-TC-3′ site (the preferred DddA target sequence context; Mok et al.) on either strand that showed the alternative sequence 5′-TT-3′ (representing cytidine deamination), and corresponding base counts and allele frequencies were tabulated using Pandas v. 1.3.0. (pandas.pydata.org).

To generate minimally filtered datasets, sites with sequence coverage of less than 15 read-pairs for that sample were ignored, as were a set of 52 sites within a phage region known to display hypervariability (Klockgether, J., et al. Pseudomonas aeruginosa Genomic Structure and Diversity. Front. Microbiol. 2, 150 (2011)). Average C⋅G-to-T⋅A transition frequency was then calculated using remaining positions for each set of quadruplicate samples per condition. To generate more stringently filtered data, sites with >95% C⋅G-to-T⋅A transition frequency in all four replicates of a given sample were considered parental SNPs and were ignored. The mean C-G-to-T⋅A transition frequency was then calculated for each position at which 3 of 4 replicate samples for a given condition exhibited at least 3 sequencing reads containing the mutation. Finally, positions were excluded for which the nearest neighboring position with an average C⋅G-to-T⋅A transition frequency >0 was within more than 100 bp. To generate the representations of the data shown in FIGS. 1A-2D, this data was further processed by the calculation of a moving average employing a 75 bp window. For statistical analyses, data passing these criteria were used except a minimum of only I read was required to contain a given mutation. Additionally, positions from any single sample were removed.

Data and Code

Sequence data associated with this study is available from the Sequence Read Archive at BioProject PRJNA748760. Computer code generated for this study is available from GitHub at github.com/marade/3DSeqTools.

Statistical Analysis

We divide the analysis into two steps: peak detection and peak-parameter inference. In the first peak-detection step, we used a canonical frequentist approach: null hypothesis testing (D. R. Cox and D. V. Hinkley, Theoretical Statistics (Chapman & Hall, 1974)) to determine the number and approximate position of the peaks in the data. Then, in a second step, we optimized the model parameters describing each peak individually using a slower but more accurate numerical Maximum Likelihood Estimation (MLE) to optimize peak parameter inference.

Biophysical Model for the Allele Frequency

Motivated by the DNA effective-concentration model (e.g. K. Rippe, P. H. von Hippel, and J. Langowski, Trends in Biochemical Sciences 20, 500 (1995)), we modeled the cell-mean allele frequency at locus j as:

$${\overline{r}}_{j} = {μ}_{0} + {\sum }_{J} {δμ}_{J} ( {x}_{j} ) , \qquad ( 1 )$$

where the first term represents the activity on nonlocalized DddA-transcription-factor fusions and the second term represents the activity, at genomic position xⱼ,for a fusions specifically bound at binding site J at genomic position [symbol] . δμ_{J} will form an allele-frequency peak around site J: it will be large when sequence j and site J are proximal and nearly zero for sequences distal to the sites.

For the functional form of the peak profile, we will again consider the DNA effective-concentration model (e.g. K. Rippe, P. H. von Hippel, and J. Langowski, Trends in Biochemical Sciences 20, 500 (1995)). We will model the peak profile as a generalized Cauchy function:

C(x|/L;a,D)≡[1+|x/L|^{D}]^{-a/D}, (2)

for D=1. In this model x≡xj− [symbol] is the genomic displacement (in bp) between sequence j and binding site J. The scaling exponent a is a model parameter that controls the rapidity with which the tails decay away from the peak. Its value is determined by chromatin structure and we expect 1<a<1:5 (K. Rippe, P. H. von Hippel, and J. Langowski, Trends in Biochemical Sciences 20, 500 (1995); J. Dekker, K. Rippe, M. Dekker, and N. Kleckner, Science 295, 1306 (2002); E. Lieberman-Aiden, N. L. et al., Science 326, 289 (2009).). The parameter L defines the width of the peak and depends both on the structure of the protein fusion as well as chromatin structure.

In practice, it will be convenient to only consider peak profile functions with local position support. We will therefore use a generalized Cauchy that is cut off at |x/L|>16:

$${C}^{′} ( \frac{x}{L} ; a , D ) ≡ \{ \begin{matrix}\frac{C ( \frac{x}{L} ; a , D ) - C ( 16 ; a , D )}{C ( 0 ; a , D ) - C ( 16 ; a , D )} , & \vert x / L \vert \leq 16 \\ 0 , & \vert x / L \vert > 16\end{matrix} , \qquad ( 3 )$$

which makes no qualitative difference to shape of the peak profile.

Our model for the mean allele frequency at locus j due to specific binding at site J is therefore:

$${δμ}_{J} ( {x}_{j} ) ≡ δμ ( {x}_{j} ; {θ}_{J} ) ≡ {I}_{J} {C}^{′} ( \frac{{x}_{j} - {ℓ}_{J}}{{L}_{J} ;} ; {a}_{J} , 1 ) , \qquad ( 4 )$$

where the parameter vector contains the following parameters:

θ_{J}=(I_{J}, [symbol] _{J},a_{J},L_{J}), (5)

and the last undefined parameter Iⱼ controls the peak profile amplitude (or height). The model results in an excellent fit to the observed allele frequency peaks.

1. Modeling of the Distribution of Allele Frequency

Our model so far describes the cell-mean allele frequency at a genetic locus, however the creation of alleles is a stochastic process. Furthermore, the cells are mutated while the culture is growing, therefore alleles that are created early, grow with the population and have a higher frequency than alleles created late in the growth process. This is the well-known jackpot phenomenon (S. E. Luria and M. Delbrück, Genetics 28, 491 (1943)). Although this type of principled analysis is possible it would require a great number of parameters and potentially experimental calibrations (Q. Zheng, Mathematical Biosciences 162, 1 (2010)). Instead, we will implement a much more tractable and practical approach to the modeling of the expected distribution of allele frequencies at a given locus: We will model rᵢ as a Gaussian random variable with a locus-specific mean (Eq. 1) and variance that is proportional to the mean.

Let the data D be defined as the set of N allele frequencies r, and genomic positions xi pairs:

D≡{(xᵢ,rᵢ)}_{i=1 . . . N}. (6)

We modeled the allele frequencies r, as a Gaussian random variable. We assume a locus-dependent mean μi and variance σ²ᵢ:

Rᵢ˜ [symbol] (μᵢ,σᵢ), (7)

where Ri is capitalized because it is being interpreted as a random variable, N is the normal distribution.

Peak Detection by Null Hypothesis Testing

1. Data Binning and Processing for Peak Detection

Since the peak features are wide compared to single basepair resolution, it is convenient to detect the peaks initially at low resolution before optimizing the peak parameters using the full resolution data. The central limit theorem guarantees that for sufficiently large bins, the r_{j′} will be normally distributed, simplifying the analysis. However, as the size of the bins grows, so does the noise from the background enzyme activity. We compromised with a bin size of 250 bp.

One important feature of the allele frequency data is that not every base is a target due to the TC-sequence specificity of DddA. We therefore binned the data using a protocol that avoided the introduction of bins weighted by the number of target sites. We divided the genome into 250 bp bins. In each bin j′, we have data indexes jϵ [symbol] . We defined the position of the binned target x_{j′} as the weighted average of the sites in that bin:

$${x}_{{j}^{′}} ≡ \frac{{x}_{j} {r}_{j}}{{r}_{j}} . \qquad ( 8 )$$

If all rⱼ were zero in the bin, the index position x, was assigned the mean of xⱼ for jϵ [symbol] _{j′}. If no positions existed in the bin, the bin was omitted from the analysis. The allele frequency for the binned data r_{j′} was equal to the mean of the rⱼ for jϵ [symbol] _{j′}.

We found that at 250 bp bin size, there was still a significant amount of salt-and-pepper noise: i.e. extremely high allele frequency at single isolated position, surrounded by background level activity. Presumably the source of this noise are jackpot events early in proliferation.

To eliminate the jackpot features we use a standard approach from image processing [R. C. Gonzalez and R. E. Woods, Digital image processing (Prentice Hall, Upper Saddle River, N.J., 2008).]. We generated median filtered allele frequency ř_{j′} by taking a median of r_{j′} using the neighborhood [j′−1; j′;j′+1]. If r_{j′} was four standard deviations above the median-filter value ř_{j′}, we replace rⱼ with the median-filtered value ř_{j′}.

Note that this binned data D′={(x_{j′}, r_{j′}({(x_{j′}, r_{j′})} is used only in peak detection and the raw (unbinned and unfiltered) data D={(xᵢ, rᵢ)} is used for model parameter refinement.

2. Alternative Approach

An alternative strategy is to leave the SNPs in the data and use a statistical test to identify them later. In practice, this approach is much slower since it involves optimizing parameters at SNP positions before eventually throwing out these features later. After trying both approaches, we advocate the filtering approach since it produces the same results with much less effort.

3. Implementing the Locus-Dependent Variance in the Test Statistic

Since the dataset is dominated by peak-free regions, we can write:

$${σ}_{i}^{2} \approx {σ}_{0}^{2} \frac{{μ}_{i}}{{μ}_{0}} , \qquad ( 9 )$$

where μ₀ are σ²₀ and mean and variance over the entire dataset and μᵢ is the locus-dependent mean (Eq. 1). In this case we know that the tails of the distribution away from the peaks look exponential and not Gaussian. If we force the likelihood to be Gaussian, this will inflate the p values. It is therefore convenient to implement the variance model in the following way:

σᵢ²→σ₀² max(1,rᵢ/μ₀). (10)

If the rᵢ>>μ0, the term in the exponent will now be linear:

$$\frac{1}{2 {σ}_{i}^{2}} {( {r}_{i} - {μ}_{0} )}^{2} \approx \frac{1}{2 {σ}_{0}^{2}} {r}_{i} {μ}_{0} , \qquad ( 11 )$$

Rather than quadratic:

$$\frac{1}{2 {σ}_{0}^{2}} {( {r}_{i} - {μ}_{0} )}^{2} \approx \frac{1}{2 {σ}_{0}^{2}} {r}_{i}^{2} . \qquad ( 12 )$$

This linear dependence matches the observed distribution which decays exponentially in r. We will use this approach for estimating the p value using the binned data for peak detection.

Another approach will be implemented for parameter approximation (see below.)

4. Implementing the Locus-Dependent Variance in the Test Statistic

The first step in the null hypothesis test is to perform a maximum likelihood estimate (MLE) of the parameter values. Since the peaks constitute a negligible fraction of the sequence, we will estimate the background mean μ₀ and variance σ²₀ using the MLE analysis in the null hypothesis and leave these fixed in all nested models. In what follows, parameters will refer only to the parameters describing the peak profiles. Each peak J will be described by Θ_{J}.

To estimate the parameters from the peak profile, we must first write the minus-log-likelihood for the normal model at the N positions:

$$- log q ( D \vert θ ) = \frac{N}{2} log 2 {πσ}_{i}^{2} + {\sum }_{i = 1}^{N} {\frac{1}{2 {σ}_{i}^{2}} [ {r}_{i} - {μ}_{i} ]}^{2} , \qquad ( 13 )$$

where the position-dependent mean μᵢ(Θ) depends on the model through the peak profile function (Eqs. 1-4) and σ²₀ is approximated using Eq. 10. We now need to minimize Eq. 13 with respect to the parameters Θ.

One difficulty here is that this statistical problem is singular: As I→0 the peak position [symbol] becomes unidentifiable (S. Watanabe, Journal of Machine Learning Research. 14, 867 (2013)). We therefore must take a brute-force approach to estimating [symbol] . We use the following steps: (i) We considered a reduced sets of positions [symbol] ϵX≡{xᵢ}_{i=1 . . . N}. We exhaustively consider a peak position at each [symbol] =xᵢ. (ii) The parameters L and a were fairly consistent between peaks since they are determined by gross-level chromatin structure. Therefore in the process of peak detection, we will assign all peak the global parameter values [symbol] →400 bp and â→1:5. (iii) The final unknown MLE parameter Î can be estimated easily since δμᵢαI and a closed-form expression can be derived for it. Since C′ has only local support, the MLE estimates can be computed rapidly.

5. Null Hypothesis Testing

The test statistic λ in the likelihood ratio test is

$$λ ( D ) ≡ log \frac{{q}_{1} ( D \vert \hat{θ} )}{{q}_{0} ( D )} . \qquad ( 14 )$$

We use the canonical Neyman-Pearson approach to hypothesis testing (D. R. Cox and D. V. Hinkley, Theoretical Statistics (Chapman & Hall, 1974)). We chose a confidence level of γ=95% (i.e. a significance level of 5%). The peak exists, i.e. we will reject the null hypothesis, if

F_{Λ}(λ)>γ, (15)

where F_{\hat{ }}is the Cumulative Distribution Function (CDF) of the test statistic λ under the null hypothesis. (Note that λ is capitalized because it is being interpreted as a random variable.)

6 Bootstrap Estimate of F_{\hat{ }}

Under the normal course of events, if the model were regular in the large sample size limit, we could use the Wilk's theorem to relate the distribution of \hat{ } under the null hypothesis to a chi-squares distribution (S. S. Wilks, The Annals of Mathematical Statistics. 9, 60 (1938)). However, the model is singular (S. Watanabe, Journal of Machine Learning Research. 14, 867 (2013)) and we must therefore estimate the distribution of the test statistic explicitly.

To compute the distribution of the test statistic, we use a stochastic simulation of the null hypothesis and then compute the empirical distribution of the test statistic. Initially we attempted to use a Gaussian random variable to simulate the null hypothesis data, however the estimated p values were too small. In retrospect, it is pretty clear that the r-distribution tails decay exponentially and therefore large rᵢ's are much more frequent than predicted by a Gaussian distribution.

In this situation, one can uses a bootstrap method to estimate the test statistic (B. Efron and R. Tibshirani, An Introduction to the Bootstrap. (Chapman & Hall/CRC, Boca Raton, F L, 1993)). There are two tractable choices: (i) the canonical bootstrap approach samples from the empirical distribution consisting of the finite set of observed background allele frequencies; (ii) A parametric bootstrap method fits the observed distribution to an empirical model and then uses the model to generate simulated data. We used the parametric bootstrap since it had the ability to sample even-more-extreme allele frequencies than were observed. We fit the distribution of Allele frequencies rᵢ for the background for the GacA data to the empirical model for random variable R:

$${p}_{R} ( r ) = {P}_{0} \cdot δ ( r ) + ⋯ + Θ ( r ) Θ ( {r}_{0} - r ) \cdot ( 1 - {P}_{0} ) \frac{{b}_{1} {b}_{2}}{b + {b}_{2}} {c}^{- {b}_{1} ( {r}_{0} - r )} + ⋯ + Θ ( r - {r}_{0} ) \cdot ( 1 - {P}_{0} ) \frac{{b}_{1} {b}_{2}}{b + {b}_{2}} {e}^{- {b}_{2} ( r - {r}_{0} )} , \qquad ( 16 )$$

where δ and Θ are the Dirac delta and Heaviside function respectively. The empirical model parameters were fit using an MLE approach:

r₀=7.2906×10⁻⁵, (17)

P₀=5.386×10⁻¹, (18)

b₁=1.2972×10⁵, (19)

b₂=8.4846×10⁻³. (20)

The fit of the empirical model to the background allele frequency is excellent (not shown).

7. Estimating the Distribution of the Test Statistic

Using the parametric-bootstrap model, we simulated the null hypothesis data D′={(x_{j′}, R_{j′})} where R_{j′}˜p_{R}. For each simulated dataset, we then computed the test statistic:

Λ≡λ(D′), (21)

which we interpret as a random variable. We generated 10⁵ samples of \hat{ }. We then use the empirical distribution of A to estimate the p values in the usual way (e.g. B. Efron and R. Tibshirani, An Introduction to the Bootstrap. (Chapman & Hall/CRC, Boca Raton, F L, 1993)).

8. Computation of the p Value

We have included a p value for each detected peak as a proxy for statistical support. The p value for test statistic λ is:

p(λ)=1−F_{Λ}(λ). (22)

Since some of peaks are extremely large, the observed test statistic is much larger than any observed in our simulations. To estimate the p values in this context, we fitted the empirical distribution F_{\hat{ }} to a Gumbel distribution since the minimization of the minus-loglikelihood over [symbol] can be reinterpreted as an extreme value problem for a random [symbol] variable in the exponential family (L. Haan and A. Ferreira, Extreme value theory: an introduction. (Springer., 2007)). The Gumbel distribution is

$${F}_{A} ( λ ) = {e}^{- {e}^{- \frac{λ - {μ}_{A}}{{σ}_{A}}}} , \qquad ( 23 )$$

where the position and scale parameters are

μ_{Λ}=0.7217, (24)

σ_{Λ}=0.2823, (25)

respectively, which we estimated using an MLE approach. For very small p we can make the following approximation:

$$log p \approx - \frac{λ - {μ}_{λ}}{{σ}_{λ}} , \qquad ( 26 )$$

by Taylor expanding the outer-most exponential around zero in Eq. 23.

9. Statistical Tests for Subsequent Nested Models

After a peak is detected by rejecting the null hypothesis, we replace the null hypothesis with the alternative hypothesis and then define a new alternative hypothesis with another putative peak. We then repeat the null hypothesis test. This procedure was repeated until no more statistically significant peaks could be detected.

Parameter Inference and Fit Refinement

[symbol]

Once the peaks were detected, we refined all four profile parameters, Θ=(I, [symbol] , a, L), for each peak by direct numerical maximum likelihood estimation for all parameters, now all defined on [symbol] . Note that this optimization is performed after peak detection. This refinement is performed on the full resolution data.

For parameter inference we will use a different approach for the scaling of the variance:

$${σ}_{i}^{2} = {σ}_{0}^{2} \frac{{μ}_{i}}{{μ}_{0}} , \qquad ( 27 )$$

since the approximation in Eq. 10 fails for the higher resolution data. For parameter optimization, the tails of the distribution are of little importance. To estimate the uncertainty in the parameters, we used the Fisher information in the usual way (e.g. D. R. Cox and D. V. Hinkley, Theoretical Statistics (Chapman & Hall, 1974)). The numerical minimization resulted in a Jacobian:

J_{αi}≡[∂_{θ}_{_{α}}δμᵢ/σᵢ,](\hat{θ}). (28)

The Fisher information is then:

I=[JJ^{T}], (29)

and therefore the predicted covariance in error is

\sqrt{δθ_{α}δθ_{β}}=[I⁻¹]_{αβ}. (30)

While illustrative embodiments have been illustrated and described, it will be appreciated that various changes can be made therein without departing from the spirit and scope of the invention.

1. A method of mapping one or more DNA-protein interactions (DPIs), comprising:
(a) contacting a double stranded DNA molecule with a target protein;
(b) coupling a double stranded DNA deaminase (DddA) to the target protein;
(c) permitting deamination of one or more cytosine residues in a domain of the double stranded DNA molecule by the DddA to provide one or more uracil residues within the domain, wherein the domain comprises a DPI site for the target protein and the double stranded DNA molecule;
(d) determining the sequence of at least a portion of the double stranded DNA molecule; and
(e) detecting the domain comprising one or more cytosine deamination events, thereby mapping the DPI site for the target protein and the double stranded DNA molecule.

2. The method of claim 1, wherein the coupling of the DddA to the target protein occurs before the contacting of step (a).

3. The method of claim 1, wherein the coupling of the DddA to the target protein occurs after the contacting of step (a).

4. The method of claim 1, wherein the double stranded DNA molecule is genomic DNA in a cell.

5. The method of claim 1, wherein the DddA comprises a DddA domain with an amino acid sequence with at least about 85% identity to SEQ ID NO:1.

6. The method of claim 1, wherein the coupling of step (b) comprises providing a fusion protein comprising a target protein domain and a DddA domain, optionally wherein the DddA domain comprises an amino acid sequence with at least about 85% identity to SEQ ID NO:1.

7. The method of claim 6, wherein the fusion protein further comprises a linker domain disposed between the target protein domain and the DddA domain.

8. The method of claim 7, wherein the fusion protein comprises an amino acid sequence with at least about 85% sequence identity to SEQ ID NO:7.

9. The method of claim 6, wherein the double stranded DNA molecule is genomic DNA in a cell that further comprises a nucleic acid encoding the fusion protein, and wherein the contacting of step (a) comprises permitting expression of the fusion protein from the nucleic acid.

10. The method of claim 1, wherein the DddA is indirectly coupled to the target protein.

11. The method of claim 10, wherein the DddA is coupled to an affinity reagent that specifically binds to the target protein.

12. The method of claim 11, wherein the double stranded DNA molecule is genomic DNA in a cell and the coupling of step (b) comprises contacting the cell with the DddA coupled to the affinity reagent and permitting the affinity reagent to specifically bind to the target protein.

13. The method of claim 12, further comprising permeabilizing the cell.

14. The method of claim 1, further comprising providing a DddA inhibitor, wherein the permitting deamination step (c) comprises removing, or depleting levels of, the DddA inhibitor.

15. The method of claim 14, wherein the DddA inhibitor is a double stranded DNA deaminase A immunity (DddAI) protein.

16-31. (canceled)

32. A fusion protein, comprising a DNA deaminase (DddA) domain and a target protein domain.

33. The fusion protein of claim 32, wherein the DddA domain comprises an amino acid sequence with at least about 85% identity to SEQ ID NO:1.

34-35. (canceled)

36. A nucleic acid encoding the fusion protein of claim 32.

37. A vector comprising the nucleic acid of claim 36, further comprising an expression promoter sequence operatively linked to the nucleic acid encoding the fusion protein.

38. A kit comprising one of:
a target protein and a DNA deaminase (DddA), optionally wherein the target protein and DddA are coupled, or optionally wherein the target protein and the DddA are separate and wherein the DddA is linked to an affinity reagent that specifically binds to the target protein.

39-41. (canceled)
